SEQUENCE LISTING (17 sequences)

SEQ ID NO: 1 - DNA, 34 bp, Artificial Sequence
  Primer
gaacaaacttctgccggtaccattctgattttta

SEQ ID NO: 2 - DNA, 36 bp, Artificial Sequence
  Primer
ttcatggtggcaatgagatctcctttccacagctga

SEQ ID NO: 3 - DNA, 32 bp, Artificial Sequence
  Primer
aatcgacaatcactgtgctatggcctatattt

SEQ ID NO: 4 - DNA, 32 bp, Artificial Sequence
  Primer
aaatataggccatagcacagtgattgtcgatt

SEQ ID NO: 5 - DNA, 30 bp, Artificial Sequence
  Primer
tgacctttcccctatagataaacactgttg

SEQ ID NO: 6 - DNA, 30 bp, Artificial Sequence
  Primer
tgtgctttcccctatagataaacactgttg

SEQ ID NO: 7 - DNA, 30 bp, Artificial Sequence
  Primer
tgacctatggcctatatttgaggaggaaga

SEQ ID NO: 8 - DNA, 30 bp, Artificial Sequence
  Primer
tgtgctatggcctatatttgaggaggaaga

SEQ ID NO: 9 - DNA, 20 bp, Rattus norvegicus
tagtgagcccttccatgaac

SEQ ID NO: 10 - DNA, 20 bp, Rattus norvegicus
cagaatctgcgacaacagtg

SEQ ID NO: 11 - DNA, 22 bp, Rattus norvegicus
gagttaatgtttgatcctggcc

SEQ ID NO: 12 - DNA, 21 bp, Rattus norvegicus
ccacccactgttggctatttt

SEQ ID NO: 13 - DNA, 20 bp, Rattus norvegicus
gtcttccgctacctaagagg

SEQ ID NO: 14 - DNA, 20 bp, Rattus norvegicus
ctgtcatctgaccagctctc

SEQ ID NO: 15 - DNA, 19 bp, Artificial Sequence
  Primer
gatatcctctgtgatgtta

SEQ ID NO: 16 - DNA, 17 bp, Rattus norvegicus
ctgacctttcccctata

SEQ ID NO: 17 - DNA, 17 bp, Rattus norvegicus
ctgacctatggcctata

(19) United States
(12) Patent Application Publication
(10) Pub. No.: US 2009/0042835 A1
(43) Pub. Date: Feb. 12, 2009

(54) COMPOSITIONS AND METHODS FOR AMELIORATING HYPERLIPIDEMIA

(76) Inventor: Roger A. Davis, Solana Beach, CA (US)

Correspondence Address: Law Office of Timothy M. Brown, P.O. Box 176, Cardiff By the Sea, CA, 92007, US

(21) Appl. No.: 11/948,997

(22) Filed: Nov. 30, 2007

Related U.S. Application Data
(63) Continuation of application No. 11/809,870, filed on Jun. 1, 2007.
(60) Provisional application No. 60/810,670, filed on Jun. 2, 2006.

(51) Int. Cl. A61K 31/695; C12Q 1/02

(52) U.S. Cl. 514/63; 435/29

(57) ABSTRACT

The invention provides compositions comprising a pharmaceutical compound having one or more Microsomal Triglyceride Transfer Protein (MTP) inhibitors that are covalently linked to one or more Liver Fatty Acid-Binding Protein (L-FABP) inhibitors. Also disclosed are methods for using the inventive pharmaceutical compositions in the treatment of hepatic steatosis and hyperlipidemia while avoiding the harmful side effects of steatorrhea.

### CROSS REFERENCE TO RELATED APPLICATION(S)

This application claims priority from U.S. Provisional Application Ser. No. 60/810,670, filed Jun. 2, 2006, and is a continuation of U.S. Utility application Ser. No. 11/809,870, filed on Jun. 1, 2007. The entire content and disclosure of these applications is incorporated herein by reference.

### BACKGROUND OF THE INVENTION

1. Field of the Invention

The present invention relates generally to medicine and the treatment of hyperlipidemia and obesity, and more particularly, to compositions and methods for ameliorating hyperlipidemia. Also disclosed are methods for identifying and making pharmaceutical compounds for the treatment of hyperlipidemia.

2. Background Information

Hyperlipidemia is a condition defined by elevated blood levels of lipids, triglycerides and cholesterol. Hyperlipidemia is also identified as dyslipidemia, to describe the manifestations of different disorders of lipoprotein, metabolism. In humans, increased blood levels of low density lipoproteins (LDL) is strongly associated with a higher risk coronary heart disease (CHD). In contrast, increased blood levels of high density lipoprotein cholesterol (HDL) is associated with a lower risk of CHD. Blood levels of triglyceride-rich lipoproteins (very low density lipoproteins: VLDL) vary in parallel with the risk of CHD, metabolic syndrome and type 2 diabetes. Lowering LDL through dietary or pharmacological therapy has been shown to decrease both morbidity and mortality associated with CHD. Lowering plasma triglycerides through dietary or pharmacological therapy has been shown to decrease the development of CHD, pancreatitis, and type 2 diabetes.

In humans, the liver is the major tissue site for the production of VLDL. While some LDL may be produced directly by the liver, a majority of LDL is derived from the metabolism of VLDL. Each VLDL and LDL particle contains a single apolipoprotein B molecule (apoB). ApoB is an essential structural component of VLDL and LDL particles via its ability to form stable spherical emulsion particles of lipids. The core of VLDL consists mainly of triglycerides, which provide energy to extrahepatic tissues via fatty acids. Following the secretion of VLDL by the liver, the core triglycerides are broken down into fatty acids which are rapidly taken up by specific receptors and fatty acid binding proteins (FABPs). Fatty acids are then either stored as triglycerides or used to produce energy and heat via oxidation. The core of LDL consists of mainly cholesteryl esters, which provide cholesterol to extrahepatic tissues mainly by binding to specific cell surface receptors. Most extrahepatic tissues can take up LDL by a tightly regulated receptor (the LDL receptor). Since under normal conditions the LDL receptor expression varies inversely with cellular cholesterol levels, LDL receptor mediated uptake of LDL does not result in the accumulation of excess cholesterol. If, however, LDL becomes modified via oxidation (usually as a result of having prolonged lifetimes in blood), the modified LDL is taken up by macrophages via receptors whose expression is not linked to cellular cholesterol levels. Excess plasma LDL levels are associated with increased lifetimes in plasma, increased oxidative modification and increased uptake by arterial wall macrophages. Uptake of oxidatively modified LDL by arterial wall macrophages initiates a cascade of events leading, to inflammation within the walls of arteries and the development of atherosclerotic lesions. Atherosclerotic lesions are the major cause of heart attack, stroke and eventually cardiac failure.

Hepatic production of apolipoprotein B-containing lipoproteins is the major pathway by which essential lipids and fat-soluble nutrients are transported to peripheral tissues for anabolic and energy requirements. Three distinct gene products: apolipoprotein B (apoB), MTP and Liver Fatty Acid-Binding Protein (L-FABP, or LFABP) share “lipid binding” structural domains, which are essential for Very Low Density Lipoprotein (VLDL) assembly/secretion.

ApoB is a uniquely large (>500 kDa), amphipathic protein essential for the assembly and secretion of triglyceride-rich VLDL. The inability of the liver and intestine to produce apoB of sufficient size (˜35 kDa) is associated with a block in the assembly and secretion of apoB-containing lipoproteins. Normally, hepatic expression of apoB is constitutive; changes in hepatic secretion of apoB-con taming lipoproteins are the result of variation in the amount of de novo synthesized apoB that is either secreted or degraded within the liver.

Microsomal Triglyceride Transfer Protein (MTP) acts as both a lipid transfer protein and as a facilitator of apoB folding and translocation. MTP facilitates the transfer of four major lipid classes (free cholesterol, phospholipids, triglycerides and cholesterol esters) to the nascent apoB-containing lipoprotein particle via a two-step process. Abrogation of one of more of these concerted MTP-dependent processes leads to co-translational degradation of nascent apoB by the proteasome.

Hepatic VLDL assembly and secretion is highly variable among individuals and sensitive to changes in nutritional state. It is induced by carbohydrate feeding and repressed by fasting. These nutritional changes in VLDL secretion are linked to sterol regulatory element binding protein (SREBP)-mediated changes in the expression levels of key lipogenic enzymes. When the rate of hepatic de novo lipogenesis is reduced (i.e. fasting), fatty acids supplied by adipose tissue can provide sufficient substrate for the glycerolipid synthesis and VLDL assembly/secretion. Variations in hepatic expression levels of both MTP and L-FABP control the flux of fatty acids into glycerolipid biosynthesis and VLDL assembly/secretion.

Since the lipid transfer activity of MTP is rate-limiting for the secretion of apoB, inhibition of MTP has been considered to be an ideal target to therapeutically ameliorate hyperlipidemia. Several studies show that reducing MTP functional expression by either treating mice with an MTP inhibitor or by MTP gene knockout markedly reduced atherosclerotic lesion formation in several animal models (e.g. Watanabe Hyperlipidemic rabbits and mice genetically modified to increase their susceptibility to atherosclerosis). These findings have led drug companies to develop MTP inhibitors as potential therapeutics for hyperlipidemia. While MTP inhibitors block hepatic lipoprotein secretion and decrease plasma lipid levels, they also cause hepatic steatosis (i.e. fatty liver development). Thus, while MTP inhibitors are effective against hyperlipidemia (a major cause of heart disease), they are not safe because they cause hepatic steatosis. Accordingly, a need exists for compounds and methods for the treatment of hyperlipidemia and obesity without causing fatty liver development.

### SUMMARY OF THE INVENTION

The present invention is based on the finding that blocking the lipid transfer activity of L-FABP (by either genetic mutation of the L-FABP gene or by adding a chemical that blocks L-FABP lipid transfer activity) allows MTP inhibitors to reduce plasma lipids without causing hepatic steatosis (i.e. the development of fatty liver). (While MTP inhibitors are effective in reducing hepatic lipid secretion, without inhibiting L-FABP, because of the associated injury caused by the development of fatty liver, the use of MTP inhibitors is either severely limited or prohibited.) This invention is based on the discovery that co-inhibition both L-FABP and MTP lipid transfer activities prevents the formation of fatty liver caused by inhibiting MTP, while still achieving the therapeutic amelioration of hyperlipidemia. Based on this discovery, the invention provides pharmaceutical compounds having the dual capability of inhibiting both L-FABP and MTP. As a result of this dual L-FABP and MTP inhibition, hepatic lipoprotein secretion is reduced, plasma lipids are reduced and the injury caused by the development of fatty liver is reduced or avoided. This invention describes how single agents having dual L-FABP and MTP inhibitory activities can be identified and used as single entities or how single agents can be assembled from one agent having L-FABP inhibitory activity and another distinct agent having MTP inhibitory activity via their covalent coupling to form a single compound agent having a distinct chemical composition.

Accordingly, an objective of the invention is to provide a pharmaceutical compound made according to the process of providing a first agent that inhibits MTP, providing a second agent that inhibits L-FABP, and forming said pharmaceutical compound by covalently linking said first agent and said second agent.

Another objective of the invention is to provide a method for identifying a candidate combination therapy for treating hyperlipidemia comprising determining a first agent that inhibits MTP, and a second agent that inhibits L-FABP, and identifying the combination of said first agent and said second agent as a candidate combination therapy treating hyperlipidemia.

Another objective of the invention is to provide a method for identifying a candidate composition for the treatment of hyperlipidemia comprising providing a test composition, determining that said test composition has the ability to inhibit MTP and L-FABP, and identifying said test composition as a candidate composition for the treatment of hyperlipidemia.

Another objective of the invention is to provide a method for identifying a candidate pharmaceutical compound for the treatment of hyperlipidemia comprising identifying a first agent that inhibits MTP, identifying a second agent that inhibits L-FABP, and identifying, as a pharmaceutical compound, a molecule formed by covalently linking said first agent and said second agent.

Another objective of the invention is to provide a method for treating hyperlipidemia comprising administering, to a patient in need thereof, an effective amount of a pharmaceutical compound that inhibits MTP and L-FABP.

Another objective of the invention is to provide a method for treating hyperlipidemia comprising administering to a patient in need thereof a pharmaceutical compound comprising a MTP inhibitor and a L-FABP inhibitor, wherein the MTP inhibitor and the L-FABP inhibitor are linked by covalent bonding in a manner that permits the pharmaceutical compound to be absorbed by the intestine intact, and achieve physiological activation in the liver.

Another objective of the invention is to provide a method for treating hyperlipidemia comprising administering, to a patient in need thereof, an effective amount of (a) a first agent that inhibits MTP, and (b) a second agent that inhibits L-FABP.

Another objective of the invention is to provide a method of screening for a pharmaceutical compound useful in the treatment of hyperlipidemia comprising the steps of (a) identifying a MTP inhibitor, (b) identifying a L-FABP inhibitor, (c) forming a pharmaceutical compound by covalently linking the MTP inhibitor and the L-FABP inhibitor in a manner that permits the pharmaceutical compound to achieve (i) intact absorption in the intestine and (ii) physiological activation in the liver, and (d) testing the ability of said pharmaceutical compound for the ability of the pharmaceutical compound to treat hyperlipidemia in vivo.

Another objective of the invention is to provide a pharmaceutical compound made by a process comprising the steps of identifying a MTP inhibitor, identifying a L-FABP inhibitor, and covalently linking said MTP inhibitor and said L-FABP inhibitor in a manner that permits the pharmaceutical compound to be administered to a patient in a defined stoichiometry.

Another objective, of the invention is to provide a method for screening for an effective stoichiometric ratio for a conjugate pharmaceutical compound useful in the treatment of hyperlipidemia, the method comprising (a) identifying a MTP inhibitor, (b) identifying an L-FABP inhibitor, (c) combining the MTP inhibitor and the L-FABP inhibitor in a pharmaceutical formulation using a selected stoichimetric ratio, and (d) testing the effectiveness of the pharmaceutical formulation's selected stoichiometric ratio in the treatment of hyperlipidemia in vivo.

Another objective of the invention is to provide a method for screening for an effective stoichiometric ratio for a conjugate pharmaceutical compound useful in the treatment of hyperlipidemia, the method, comprising (a) identifying a MTP inhibitor, (b) identifying an L-FABP inhibitor, (c) forming the pharmaceutical conjugate by covalently linking the MTP inhibitor and the L-FABP inhibitor using a selected stoichimetric ratio, and (d) testing the effectiveness of the pharmaceutical conjugate's selected stoichiometric ratio in the treatment of hyperlipidemia in vivo. (For example, it may be useful to formulate a single orally administered molecule that contains 2 molecules of a L-FABP inhibitor and one molecule of the MTP inhibitor covalently linked to glycerol via carboxylate ester bonds. Alternatively, it might be useful to formulate a single orally administered molecule that contains 1 molecule of a L-FABP inhibitor and 2 molecules of the MTP inhibitor covalently linked to glycerol via carboxylic ester bonds.)

Another objective of the invention is to provide a method testing the liver-specific activation of a pharmaceutical compound useful in the treatment of hyperlipidemia comprising (a) providing a MTP inhibitor, (b) providing a L-FABP inhibitor, (c) forming the pharmaceutical compound by linking the MTP inhibitor and the L-FABP inhibitor by covalent bonding, (d) administering the pharmaceutical composition in vivo, (e) measuring its rate of absorption by the intestine, it concentration in blood, its concentration in liver and other tissues and its ability to alter plasma and liver lipids.

Empirical analysis of how changes in the structure of the L-FABP and MTP inhibitor and the covalent bond used to produce the single pharmaceutical agent affect each of these parameters will allow the identification of specific dual L-FABP/MTP inhibitors having distinct physiological endpoints in regard to intestinal absorption, intestinal metabolism, hepatic uptake, hepatic metabolism and effects on hepatic and plasma lipids. Our invention targets liver L-FABP and MTP inhibition in a manner to avoid intestinal L-FABP and MTP inhibition, which is associated with the undesirable side-effects of steatorrhea. Identifying single covalently linked complexes composed of a L-FABP inhibitor and a MTP inhibitor having distinct tissue (liver versus intestine) selective effects would provide enhance therapeutic utility.

Another objective of the invention is to provide a pharmaceutical compound for the treatment of hyperlipidemia comprising a molecule that inhibits MTP and L-FABP.

Another objective of the invention is to provide a pharmaceutical compound for treating hyperlipidemia comprising a MTP inhibitor and a L-FABP inhibitor, wherein the MTP inhibitor and the L-FABP inhibitor are linked by covalent bonding.

Another objective of the invention is to provide a pharmaceutical compound useful in the treatment of hyperlipidemia comprising a first pharmaceutical agent that inhibits MTP, and a second pharmaceutical agent that inhibits L-FABP, wherein the first and second pharmaceutical agents are linked by covalent bonding.

Another objective of the invention is to provide a kit for the treatment of hyperlipidemia comprising at least one MTP inhibitor, and at least one L-FABP inhibitor, wherein said MTP inhibitor and said L-FABP inhibitor are assembled using a packaging material.

The invention also provides a method of screening for an inhibitor of MTP and L-FABP activity, comprising contacting a cell expressing MTP and L-FABP with at least one test agent and detecting decreased expression of MTP and L-FABP following contact, wherein detection of decreased expression of MTP and L-FABP following contact identifies the agent as an inhibitor of MTP and L-FABP.

The details of one or more embodiments of the invention are set forth in the accompanying drawings and the description below. Other features, objects, and advantages of the invention will be apparent from the description and from the claims.

### BRIEF DESCRIPTION OF THE DRAWINGS

FIGS. 1A-1C are graphical diagrams showing that MTP and L-FABP demonstrate similar cell-type specific differences in both mRNA and promoter activity levels.

FIG. 1A shows the conserved DR1 elements (5′ and 3′ hexameric half-sites are underlined) within proximal regions of both MTP (SEQ ID NO: 16) and L-FABP (SEQ ID NO: 17) promoters (rat) are provided.

FIG. 1B shows results of sybr green real time PCR analysis of MTP and L-FABP mRNA levels in 1.35 and FAO cells. All values normalized to levels of 36B4 mRNA.

FIG. 1C shows that luciferase constructs driven by either the MTP (−135/+66) or L-FABP (−141/+66) promoters were transiently transfected into L35 and FAO cells. Constructs containing mutant DR1 elements consist of base pair changes in the 5′ hexameric half sites of each promoter from AC to TG as detailed below. Luciferase activities are represented by filled bars (FAO cells) and empty bars (L35 cells). All luciferase values were normalized to a Renilla control. Error bars indicate S.D. of triplicate samples.

FIGS. 2A and 2B are pictorial diagrams showing that COUP-TFII binds to the proximal DR1 site acting as a repressor of L-FABP promoter activity.

FIG. 2A shows that utilizing EMSA with a radiolabeled L-FABP-DR1 probe, differential complexes were attained comparing nuclear extracts from L35 and FAO cells. To assess the presence of COUP-TFII in the L35-/FAO-specific complexes, antibodies specific for COUP-TFII were added to the nuclear extract during incubation with the L-FABP-DR1 probe. The L35-specific COUP-TFII complexes with and without antibody addition, are indicated by a filled arrow and open arrow, respectively.

FIG. 2B shows that cotransfection of COUP-TFII in FAO cells decreases L-FABP promoter activity. A luciferase reporter plasmid containing sequences −141/+66 of the rat L-FABP promoter was cotransfected with the indicated amounts of COUP-TFII expression plasmid into FAQ cells. The construct containing the mutant DR1 element is as described in FIG. 1. All luciferase values were normalized to a Renilla control. Error bars indicate S.D. of triplicate samples.

FIGS. 3A-3C are pictorial and graphical diagrams showing that cell-type specific complex formation with the DR1 element in L35 (COUP-TFII) and FAO (PPARα/RXRα) cells reflect the relative expression ratio of the nuclear receptors COUP-TFII:PPARα/RXRα.

FIG. 3A shows that utilizing EMSA with a radiolabeled L-FABP-DR1 or MTP-DR1 probes, similar FAO-specific complexes (filled arrows) were attained. To assess the presence of PPARα/RXRα in the FAO-specific complexes, antibodies specific for PPARα (P), RXRα (R), or COUP-TFII (C) were added to the nuclear extract, during incubation with either the L-FABP-DR1 or MTP-DR1 probe.

FIG. 3B shows the results from ChIP assays comparing L35 and FAO cells, performed utilizing antibodies specific for COUP-TFII and PPARα. Relative amounts of region specific DNA were determined by real time PCR using primers specific for either L-FABP-DR1 or MTP-DR1 promoter regions (filled bars) as indicated. Relative levels of distal untranslated regions (open bars) are given to demonstrate region, specificity. All values were normalized to immunoprecipitations with IgG as described below.

FIG. 3C shows the results from real time PGR analysis of COUP-TFII, RXRα, and PPARα mRNA levels in L35 (open bars) and FAO (filled bars) cells. All values normalized to levels of 36B4 mRNA. Error bars indicate S.D. of triplicate samples.

FIGS. 4A-4C are graphical and pictorial diagrams showing that PPARα/RXRα agonist treatment of L35 cells allows for the coordinate induction of L-FABP and MTP mRNAs, DR1 site-dependent increased promoter activity levels, and a restored ability for apoB secretion.

FIG. 4A shows the results from real time PCR to determine relative L-FABP and MTP mRNA levels comparing untreated L35 cells (−) to those treated for 48 hours with either the PPARα agonist WY-14,643 (WY), the RXRα 9-cis retinoic acid (RA) agonist, or the vehicle (DMSO) as indicated. WY/RA indicates L35 cells treated with both agonists simultaneously. All values were normalized to levels of 36B4 mRNA.

FIG. 4B shows that utilizing both the wild type and mutant-DR1 luciferase reporter constructs (described in FIG. 1), relative promoter activity levels, for both L-FABP and MTP, were determined comparing untreated L35 cells (−) to those treated for 48 hours as described above. Promoter activities axe indicated for wild type as open bars and for the mutant-DR1 constructs as filled bars. All luciferase values were normalized to a Renilla control. Error bars indicate S.D. of triplicate samples.

FIG. 4C shows that L35 cells were cultured in the absence (lanes 1-3) or presence (lanes 4-6) of 1 μM 9-cis retinoic acid and 10 μM WY-14,643 for 72 hrs before being labeled with [³⁵S]-methionine. Media was collected 24 hrs after the addition of radioactivity. Secreted apoB was immunoprecipitated with a polyclonal anti-apoB antibody and resolved by SDS-PAGE (4-12%). Labeled proteins were detected by autoradiography. The locations of apoB48 and apoB100 was determined by molecular weight markers and human LDL standards.

FIGS. 5A and 5B are graphical diagrams showing that PPARα/RXRα agonist, treatment of L35 cells reduces the nuclear receptor ratio of COUP-TFII:PPARα/RXRα resulting in altered occupancy of the proximal-DR1 region of both L-FABP and MTP promoters from the repressive COUP-TFII complex to an activating PPARα/RXRα complex.

FIG. 5A shows that using real time PCR, relative mRNA levels of COUP-TFII, RXRα, and PPARα were determined comparing untreated L35 cells (open bars) to those treated for 48 hours with WY-14,643 and 9-cis retinoic acid simultaneously (filled black bars) or DMSO (filled grey bars) as indicated. All values were normalized to levels of 36B4 mRNA.

FIG. 5B shows the results from ChIP assays comparing untreated L35 and FAO cells to L35 cells treated with both WY-14,643 and 9-cis retinoic acid (L35W/R), which were performed utilizing antibodies specific for COUP-TFII and PPARα as indicated. Relative amounts of region specific DNA was determined by real time PCR using primers specific for either L-FABP-DR1 or MTP-DR1 promoter regions (filled bars) as indicated. Relative levels of distal untranslated regions (open bars) are given to demonstrate region specificity. All values were normalized to input DNA and immunoprecipitations with IgG as described in “Experimental Procedures,” Error bars indicate S.D. of triplicate samples.

FIGS. 6A and 6B are graphical diagrams showing that PPARα is necessary for maintenance of L-FABP and MTP expression in FAO cells and for the GW-7647 mediated induction of both genes in vivo.

FIG. 6A shows the results of RNA interference knockdown of PPARα, which was achieved by transfecting FAO cells for 72 hours with either PPARα-specific siRNAs or non-targeting control siRNAs as a negative control. Using real time PCR, relative mRNA levels of PPARα, MTP, L-FABP, and ApoB were determined comparing FAO cells treated with PPARα-specific siRNA (filled bars) to those treated with the negative control siRNA (open bars). The mRNA levels in FAO cells treated with PPARα-specific siRNAs are expressed as percentages of the negative Control set to 100%. All values were normalized to levels of 18S mRNA.

FIG. 6B shows that control C57BL/6 and PPARα−/− mice (5 mice/group) were treated with the PPARα agonist GW-7647 for 7 weeks. Using real time PCR relative mRNA levels of L-FABP and MTP were determined. Relative mRNA levels are, represented as filled bars (GW-7647 treated) and open bars (vehicle treated). All values were normalized to levels of 18S mRNA. Error bars indicate S.D. of triplicate samples.

FIGS. 7A-7C are graphical diagrams showing that PGC-1β expression correlates with L-FABP and MIT both in hepatoma cells and in vivo, and is necessary for PPARα/RXRα induced expression of both genes of both genes in FAO cells.

FIG. 7A shows the results of real time PCR analysis of PGC-1α and PGC-1β mRNA levels in L35 (open bars) and FAO cells (filled bars). All values normalized to levels of 36B4 mRNA.

FIG. 7B shows the results of real time PCR analysis of PGC-1β mRNA levels comparing untreated L35 (open bars) and L35 cells treated with WY-14,643 and 9-cis retinoic acid simultaneously (filled bars) for 48 hours. Additionally, PGC-1β mRNA levels were determined comparing untreated wild mice (Wt, open bars) and wild type mice treated with the PPARα agonist GW-7647 for 7 weeks (Wt GW-7647, filled bars). All values normalized to levels of 36B4 mRNA.

FIG. 7C shows the results of RNA interference knockdown of PGC-1β, which was achieved by transfecting FAO cells for 72 hours with either PGC-1β specific siRNAs or non-targeting siRNAs as a negative control. 48 hours prior to harvesting, cells were treated with WY (10 μM) and RA (1 μM). Using real time PCR, relative mRNA levels of PGC-1β, MTP, L-FABP, and PGC-1α were determined comparing FAO cells treated with PGC-1β-specific siRNA (filled bars) to those treated with the negative control siRNA (open bars). The mRNA levels in FAO cells treated with PGC-1β-specific siRNAs are expressed as percentages of the negative control set to 100%. All values normalized to levels of 36B4 mRNA. Error bars indicate S.D. of triplicate samples.

FIG. 8 is a graphical diagram showing that PGC-1β-mediated increase of L-FABP and MTP in L35 cells is PPARα/RXRα-dependent. Real time PCR analysis of L-FABP and MTP mRNA levels in L35 cells treated with adenoviral constructs and agonists as indicated for 48 hours. L35 cells; were infected with either Ad-PGC-1β, Ad-GFP or uninfected as indicated. Infection coincided with simultaneous agonist treatment (WY-14,643 and 9-cis retinoic acid) for 48 hours. All values (mean±S.D. of triplicate samples) were normalized to levels of 36B4 mRNA.

FIGS. 9A and 9B are graphical diagrams showing that prevention of the MTP inhibitor induced hepatic steatosis by ablation of the L-FABP gene. Male C57BL/6 and L-FABP−/− mice (6 mice/group) were given the MTP inhibitor 8aR (50 mg/day/kg) for 7 days. Mice were sacrificed, plasma and livers were obtained and lipid levels determined.

FIG. 9A shows that plasma triglyceride and cholesterol levels were determined for both C57BL/6 and L-FABP−/− mice comparing vehicle treated (open bars) to those treated with 8aR (filled bars).

FIG. 9B shows that liver triglyceride levels were measured for both strains comparing untreated (open bars) to those treated with 8aR (filled bars). All values represent the mean±SD.*P<0.001.

FIG. 10 is a graphical diagram showing that co-administration of a L-FABP inhibitor with a MTP inhibitor reduces plasma triglyceride concentrations in mice.

FIG. 11 is a graphical diagram showing that co-administration of a L-FABP inhibitor with a MTP inhibitor reduces plasma cholesterol concentrations in mice.

FIG. 12 is a graphical diagram showing that co-administration of a L-FABP inhibitor with a MTP inhibitor prevents the development, of hepatic steatosis in mice.

### INCORPORATION BY REFERENCE

All publications and patents mentioned in this specification are herein incorporated by reference to the same extent as if each individual publication or patent was specifically and individually indicated to be incorporated by reference.

### DEFINITIONS

Unless defined otherwise, all technical and scientific terms used herein, have the same meaning as commonly understood by one of ordinary skill in the art to which this invention belongs. Although any methods and materials similar or equivalent to those described herein can be used in the practice or testing of the invention, the preferred methods and materials are now described.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “pharmaceutical agent” refers to a substance that provides a desired effect when administered to the body of a subject. A pharmaceutical active may also be referred to as a drug. The pharmaceutical actives of the invention may assume the form of small molecules (having a molecular weight of more than 100 and less than about, 2,500 daltons), peptides, carbohydrates, lipids, or a combination thereof. A substance, such as a small molecule, that inhibits MTP and/or L-FABP is one long limiting example of a pharmaceutical agent.

The expression “pharmaceutical compound,” or “compound,” refers to a molecule that has a desired effect when administered to the body of a subject. Such pharmaceutical compounds include, but are not limited to, small organic molecules (i.e. molecules having a molecular weight of more than 100 and less than about 2,500 daltons). Pharmaceutical compounds include small molecules which inhibit one or both of MTP and L-FABP. Pharmaceutical compounds may be conjugated or non-conjugated. Conjugated pharmaceutical compounds are formed by covalently bonding a MTP inhibitor a L-FABP inhibitor, while non-conjugated pharmaceutical compounds are formed a single molecule that has dual MTP and L-FABP inhibitory activity.

The term “agent” or “pharmaceutical agent” refers to a substance that has the ability to inhibit either MTP or L-FABP. The agents of the invention include, but are not limited to, small organic molecules which are covalently linked (i.e. bonded to one another) to form a pharmaceutical compound having dual MTP and L-FABP inhibitor activity.

The term “co-drug” refers to a pharmaceutical compound which comprises one or more MTP inhibitors and one or more L-FABP inhibitors which are joined by covalent bonding.

The expression “pro-drug” refers to compounds that are drug precursors which following administration, release the drug in vivo via some chemical or physiological process (e.g., a pro-drug on being brought to the physiological pH or through enzyme action is converted to the desired drug form).

The term “treating,” “treat” or “treatment” as used herein includes preventative (e.g., prophylactic) and palliative treatment.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The term “pharmaceutically acceptable”, when used in reference to a carrier, is meant that the carrier, diluent or excipient must be compatible with the other ingredients of the formulation and not deleterious to the recipient thereof. The term “pharmaceutically acceptable” as used herein means suited for normal pharmaceutical applications, i.e. giving rise to no adverse events in patients etc.

The terms “administration” or “administering” are defined to include an act of providing a compound or pharmaceutical composition of the invention to a subject in need of treatment. The phrases “parenteral administration” and “administered parenterally” as used herein means modes of administration other than, enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The term “agonist” refers to an agent or analog that binds productively to a receptor and mimics its biological activity. The term “antagonist” refers to an agent that binds to receptors but does hot provoke the normal biological response. Agonists or antagonists may include proteins, nucleic acids, carbohydrates, antibodies, or any other molecules which decrease the normal biological response.

The term “antibody” as used in this invention is meant to include intact molecules of polyclonal or monoclonal antibodies, as well as fragments thereof, such as Fab and F(ab′)₂, Fv and SCA fragments which are capable of binding an epitopic determinant.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined. In another aspect, the corresponding normal cells comprise a sample of cells obtained from an otherwise healthy portion of tissue of a subject having hyperlipidemia.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

As used herein, the terms “reduce” and “inhibit” are used to refer to a substance (e.g. small molecule) that has the ability to decrease the rate or overall amount of a biological process by a level that can be analytically measured. MTP and L-FABP enzymatic activity, both in vivo and in vitro, are examples of a biological process that may be “reduced” or “inhibited.” MTP and L-FABP production, both in vivo and in vitro, are also examples of a biological process that may be “reduced” or “inhibited.”

As used herein, the term “steatosis” refers to the process describing the abnormal retention of lipids within a cell. It reflects an impairment of the normal processes of synthesis, transport and breakdown of lipids, usually triglycerides and cholesteryl esters. Excess lipid accumulates in intracellular membrane and in vesicles that displace the cytoplasm. When the vesicles are large enough to distort the nucleus, the condition is known as macro-vesicular steatosis, otherwise the condition is known as microvesicular steatosis. Whilst not particularly detrimental to the cell in mild cases, large accumulations can alter membrane structure and impair membrane function and cell viability. As such, “steatosis” includes the accumulation of fat in the interstitial tissue of an organ. The risk factors associated with steatosis are varied, and include the production of inflammatory lipids and cytokines. Proinflammatory lipids and cytokines promote atherosclerosis, diabetes mellitus, malignancy, hypertension, cell toxins, obesity, and anoxia. As the liver is the primary organ of lipid metabolism it is most often associated with steatosis, however it may occur in any organ, commonly the kidneys, heart, and muscle. Hepatic steatosis enhances the development of severe liver diseases such as cirrhosis and hepatocellular carcinoma.

As used herein the term “cytokine” encompasses chemokines, interleukins, lymphokines, monokines, colony stimulating factors, and receptor associated proteins, and functional fragments thereof. Exemplary cytokines include, but are not limited to, endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration, in a cell or cell mechanism.

### DETAILED DESCRIPTION OF THE INVENTION

The present invention, is generally relates to the discovery that co-inhibition of Microsomal. Triglyceride Transfer Protein (MTP) and Liver Fatty Acid-Binding Protein (L-FABP, or LFABP) can ameliorate hyperlipidemia without causing hepatic steatosis (“fatty liver”), thus providing an effective and safe way to treat hyperlipidemia. Based on this discovery, the present invention provides compositions, screening assays, and methods of treatment for addressing hyperlipidemia. The invention further provides methods for formulating and administering MTP and L-FABP inhibitors in pharmaceutical compositions that avoid intestinal L-FABP and MTP inhibition which leads to harmful steatorrhea.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Fat is transported from the intestine and liver through blood in the form of lipoproteins. The production of lipoproteins by both the liver and intestine require an unusually large amphipathic protein apolipoprotein B (apoB). ApoB is secreted by the liver as the structural, protein in triglyceride-rich very low density lipoproteins (VLDL). In plasma, the triglycerides carried in VLDL are removed as fatty acids and delivered to extrahepatic tissues for use in energy production and anabolism. The particle containing apoB is converted to LDL. Increased plasma levels of cholesterol-rich low density lipoproteins (LDL) are associated with the development of atherosclerosis, the major cause of cardiovascular disease and death in the United States and other industrialized countries. The liver is the major organ site responsible for both the production and degradation of LDL. Since patients with the recessive genetic disorder abetalipoproteinemia have almost no LDL in their plasma and display reduced susceptibility to atherosclerotic cardiovascular disease, a great deal of effort has been directed toward developing therapeutic treatments that would reduce plasma LDL levels. This proposal was brought to reality by the development of drugs (i.e., statins) which have been shown to significantly reduced both morbidity and mortality from cardiovascular disease by lowering plasma LDL levels as a consequence of increased hepatic LDL uptake and degradation. Not all people can safely take statins, providing an impetus toward developing additional drugs to reduce plasma LDL levels.

The discovery that abetalipoproteinemia is caused by a mutational loss of functional, microsomal triglyceride transfer protein (MTP) suggested MTP-lipid transfer is required for the assembly and secretion of apoB-containing lipoproteins. As used herein, “abetalipoproteinemia” refers to a rare inherited disorder of fat metabolism due to a mutation in the MTP gene causing a functional loss in MTP function. MTP is a protein present in the endoplasmic reticulum (ER) as a complex with protein disulfide isomerase (PDI). MTP was originally identified as a protein that facilitated the transfer of lipids (triglycerides, phospholipids, cholesterol and cholesterol esters) from membranes to lipoproteins. MTP plays two roles in the assembly of apoB containing lipoproteins: (1) it allows apoB to be translocated into the lumen of endoplasmic reticulum by facilitating the transfer of lipids to apoB and (2) it facilitates apoB folding. In the absence of MTP activity, apoB is efficiently degraded in the endoplasmic reticulum. As a result of functional inactivation of MTP, the liver of patients having abetalipoproteinemia can not synthesize nor secrete apoB-containing lipoproteins causing levels of plasma LDL (previously designated as beta-lipoproteins) to decrease to almost undetectable levels (i.e. abetalipoproteinemia). Thus, it was reasoned that drugs that blocked MTP activity might be useful for reducing plasma LDL levels.

Since in general abetalipoproteinemics neither produce hepatic apoB-containing lipoproteins due to the absence, of MTP, nor do they exhibit fatty liver, it was reasoned that there must be a compensatory process preventing the development of steatosis in the livers of abetalipoproteinemics. To clarify this issue a hepatocyte culture model (L35 cells) that lacks the expression of MTP, but does not accumulate fat was examined. The present invention therefore shows that the transcription of the MTP gene was coordinately regulated with another gene controlling the uptake of fatty acids by the liver (L-FABP) via the presence of similar transcriptional DR1 promoter elements. Since MTP and L-FABP are thought to have been derived from a common ancestral (1,2) the retention of similar DR1 promoter elements suggested that coordinate transcription expression was physiologically important. Subsequent experiments showed that coordinate repression of MTP and L-FABP could explain why the L35 cells did not accumulate fat in the absence of MTP-facilitated lipoprotein secretion. In the absence of L-FABP, inhibition of MTP reduces hepatic VLDL secretion without causing fat accumulation (steatosis) because L-FABP facilitated uptake of fatty acids is lost.

While FAO rat hepatoma cells express L-FABP and MTP and assemble and secrete VLDL, L35 cells, derived as a single cell clone from FAO cells, neither express L-FABP or MTP nor do they assemble and secrete VLDL. As such, hepatoma cells were used to elucidate how a conserved DR1 promoter element present in the promoters of L-FABP and MTP affects transcription, expression and VLDL production. In FAO cells, the DR1 elements of both L-FABP and MTP promoters are occupied by PPARαRXRα, which with PGC-1β, activates transcription. In contrast, in L35 cells the DR1 elements of both L-FABP and MTP promoters are occupied by COUP-TFII and transcription is diminished. The combined findings indicate that PPARαRXRα and PGC-1 [symbol] coordinately upregulate L-FABP and MTP expression, by competing with COUP-TFII for the DR1-sites in the proximal promoters of each gene. Additional studies show that ablation of L-FABP prevents hepatic steatosis caused by treated mice with an MTP inhibitor. Coordinated transcriptional variation, of L-FABP and MTP adapts fatty acid entrance into VLDL secretion without causing hepatic steatosis.

Thus, the data provided herein shows that the transcription of L-FABP and MTP is regulated by competitive binding to similar DR1 elements by either the fatty acid ligand-activated transcription factors (PPARα-RXRα) or COUP-TFII. As such, expression of the two lipid transfer proteins, L-FABP and MTP, which function in concert with each other, can be coordinately regulated in response to the availability of tatty acid substrate. There are many functionally distinct pathways competing for the utilization of fatty acids by the liver. These include cellular uptake, esterification in the production of other lipids (e.g. glycerolipids and cholesterol esters), β-oxidation, storage and export mainly in the form of VLDL lipids. The delivery of fatty acids into one or more of these pathways must vary rapidly and selectively in order to maintain energy and substrate homeostasis. Substrate-driven “feed-forward” transcriptional, regulation is a common mechanism that allows both homeostasis and efficient and appropriate utilization to occur concomitantly (8). Since fatty acids also can activate PPARα-dependent gene transcription of L-FABP (9) and MTP (10), fatty acid flux to the liver both induces the enzymes controlling VLDL assembly/secretion as well as providing lipogenic substrate.

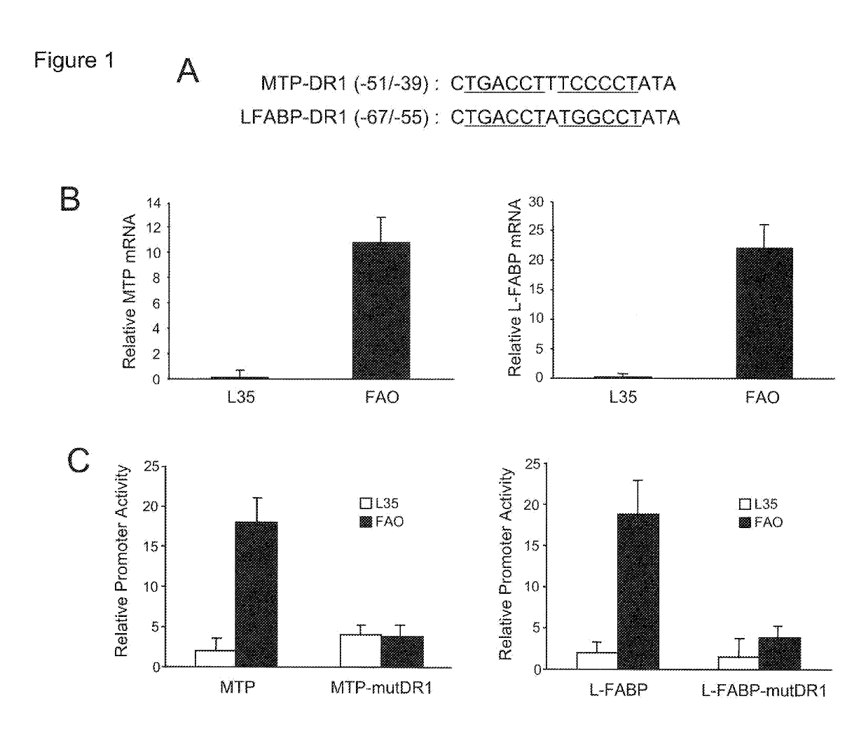

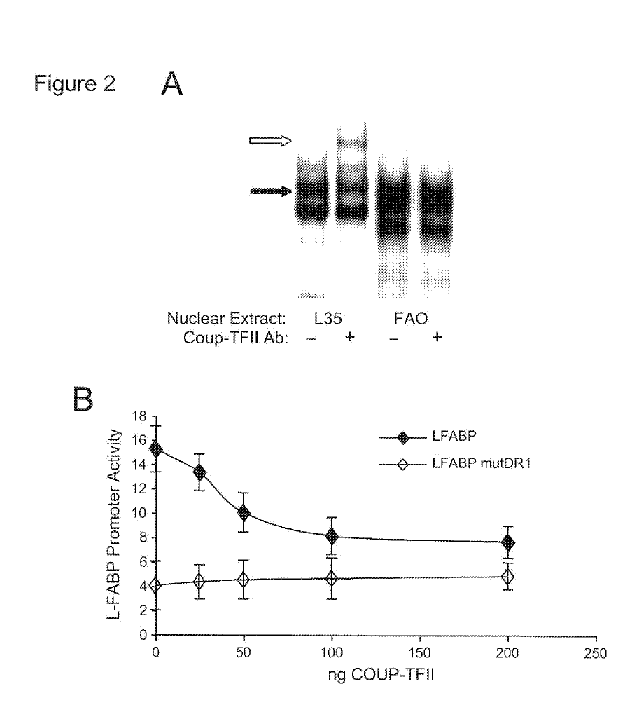

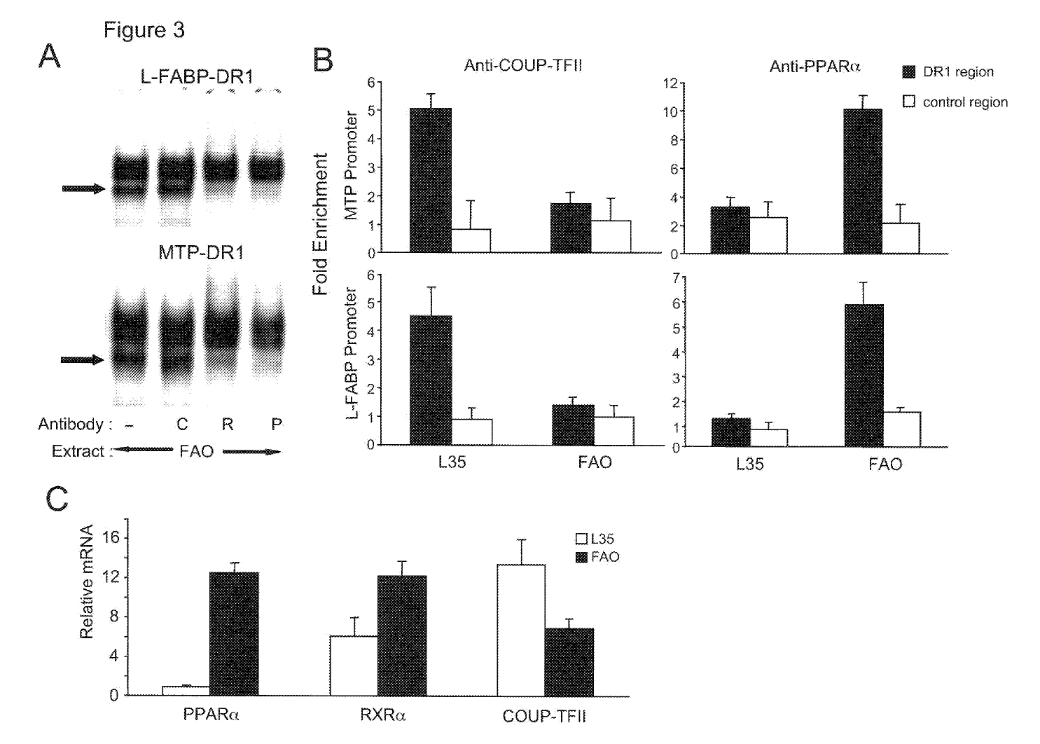

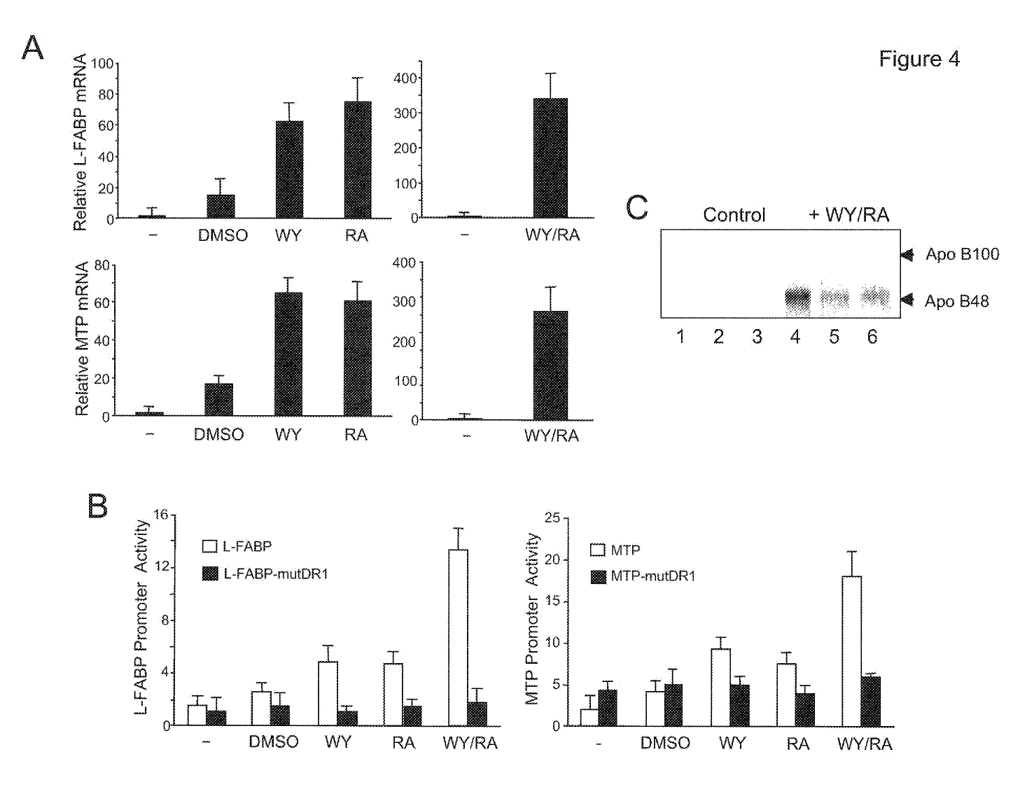

Several lines of evidence indicate that similar DR1 elements present in the L-FABP and MTP promoters provide coordinate transcriptional regulation necessary for the interdependent role of these two lipid transfer proteins in delivering fatty acids to the VLDL assembly/secretion pathway. Mutational deletion of the DR1 element, in both the L-FABP and MTP promoter reporter constructs, caused: (1) a reduction of the relatively high activity levels exhibited by FAO cells to the lower levels exhibited by L35 cells (FIG. 1C); and, (2) abrogation of both the ability of COUP-TFII (FIG. 2B) and PPARα-RXRα agonist (FIG. 4A) to repress or activate transcription, respectively. Further evidence showing that these DR1 elements are the functionally relevant cognate binding sites responsible for competitive occupation by COUP-TFII (repressor) or PPARα-RXRα (activator) are provided by EMSA supershift ((FIG. 2-L-FABP) and (4)-MTP) and ChIP (FIG. 3) analyses. The combined analyses obtained from the complementary EMSA supershift and ChIP experiments of the DR1 elements concordantly indicate that occupation by PPARα-RXRα is associated with transcriptional activation of both genes (FAO cells), while occupation by COUP-TFII is associated with repression (L35 cells).

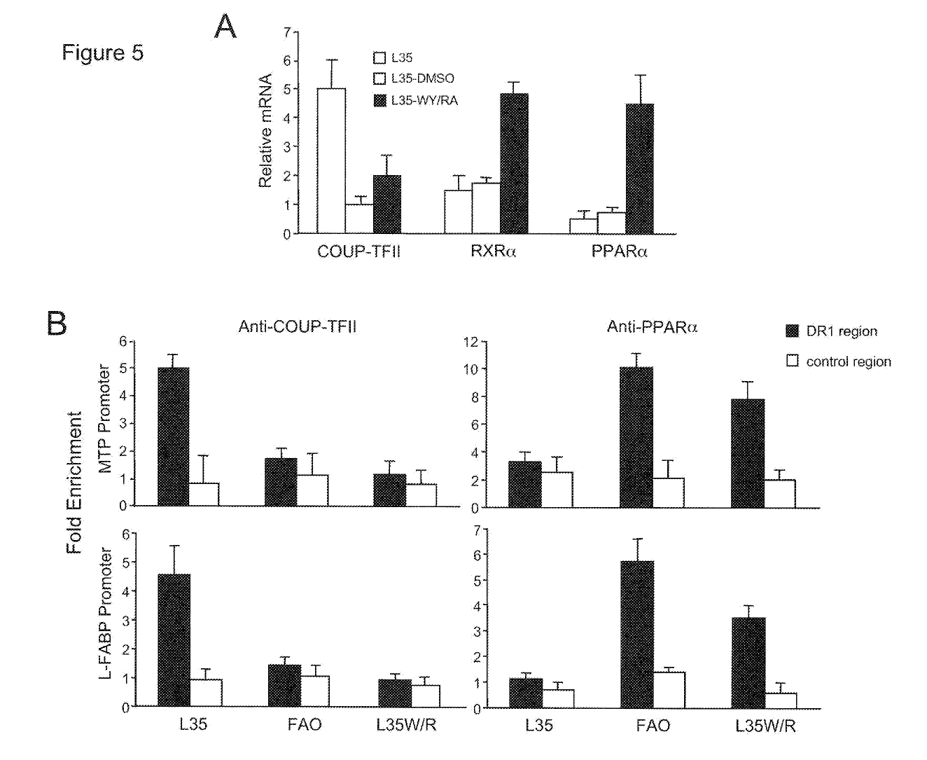

The principal determinant responsible for the phenotypic difference in the expression of L-FABP and MTP exhibited by FAO and L35 cells is the relative cellular content of COUP-TFII (repressor) to PPARα-RXRα (activator) (FIG. 3C). Three independent experiments demonstrate that the plasticity in the cellular phenotype of FAO and L35 cells is dependent upon the cellular content of COUP-TFII (repressor) relative to PPARα-RXRα (activator): (1) treating L35 cells with PPARα-RXRα agonists increases the expression of PPARα-RXRα (activator), while decreasing COUP-TFII (repressor) expression (FIG. 5A); (2) these changes in cellular content of PPARα-RXRα/COUP-TFII are reflected by similar changes in the occupancy of the DR1 elements present in both the L-FABP and MTP promoters (FIG. 5B); (3) which resulted in DR1 site-dependent increases in the transcriptional activities of both L-FABP and MTP promoter-luciferase reporter constructs (FIG. 4B); and (4) enhanced expression of L-FABP and MTP mRNAs (FIG. 4A). Furthermore, the agonist-mediated changes in L35 cells were associated with a restored ability to assemble and secrete apoB-containing lipoproteins (FIG. 4C).

PPARα-RXRα heterodimers have been shown to activate the transcription of L-FABP by binding to the DR1 site in the proximal promoter region (11). Treating wild type, but not PPARα knockout, mice with a PPARα agonist increased hepatic expression of MTP (10). These and additional findings obtained from studies examining transcriptional regulation of genes involved in fatty acid metabolism (12-35) support the proposal that PPARα-RXRα and COUP-TFII compete with each other for binding to DR1 promoter elements. In the context of these genes, occupation, of these elements by PPARα-RXRα is associated with activation of transcription, whereas occupation by COUP-TFII is associated with transcriptional repression.

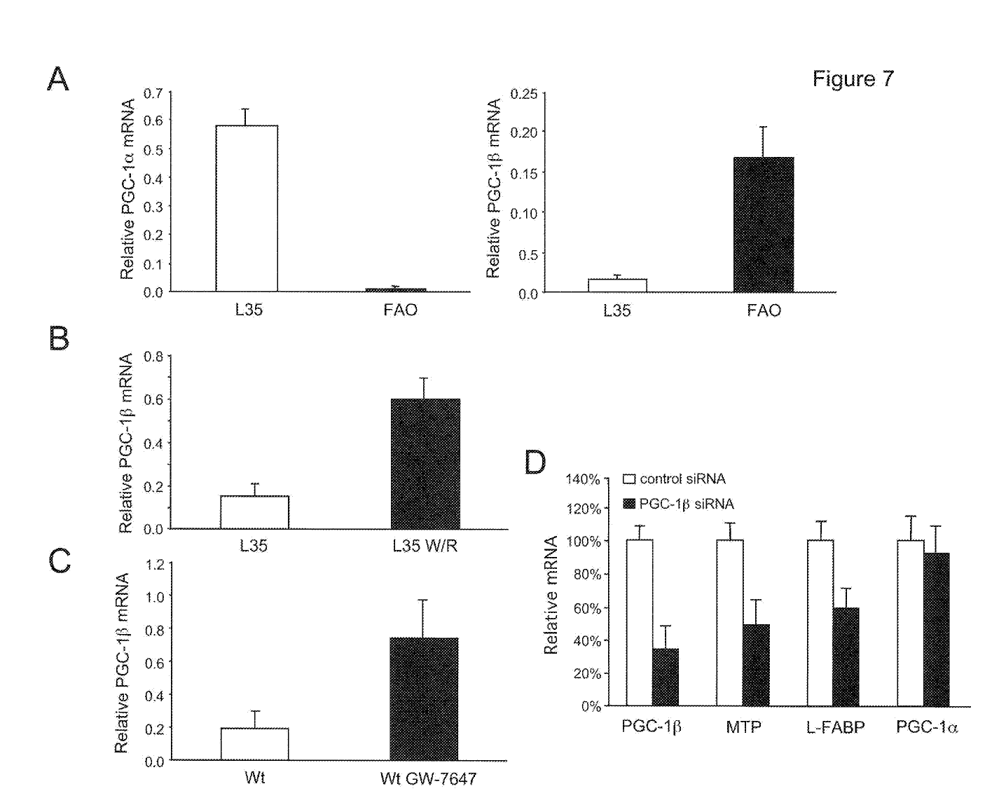

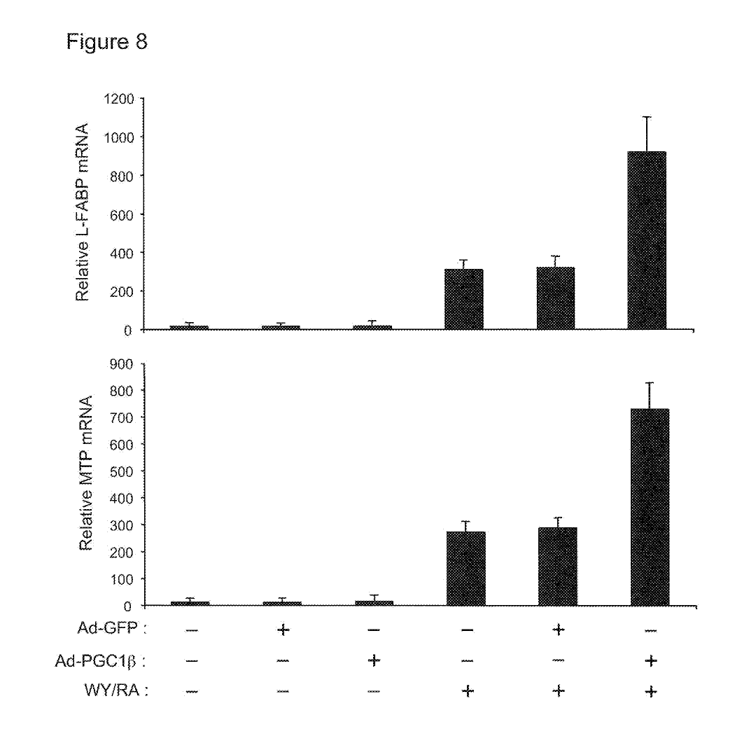

The findings disclosed herein show PGC-1β is required for PPAR [symbol] agonist-mediated induction of L-FABP and MTP expression (FIG. 7C). It was also found that while PPARα agonist treatment of PPARα−/− mice failed to induce L-FABP and MTP mRNA expression (FIG. 4), hepatic expression of PGC-1 [symbol] mRNA was increased 2-fold. These data suggest that in the absence of PPAR [symbol] PGC-1β is not sufficient to increase the expression of L-FABP and MTP. Additional experiments using adenovirus mediated expression of PGC-1β indicate that PPARα-RXRα is necessary in order to enhance the transcription of L-FABP and MTP. The combined findings support the proposal that PGC-1β participates in the transcriptional activation of MTP (16, 17). In ob/ob, diabetic mice, adenovirus mediated expression of PGC-1β and Foxa2 induced MTP expression, suggesting a mechanism through which insulin blocks VLDL assembly/secretion (17). It has been demonstrated that Foxa2 is completely excluded from the nucleus of ob/ob mice (18), while both MTP expression and VLDL assembly/secretion are increased (19). Furthermore, PGC-1β-mediated increase in MTP expression was retained in the ob/ob mice indicating that Induction of MTP transcription can occur via Foxa2 independent mechanisms (17). In fat-fed hyperlipidemic mice, PGC-1β activation of SREBP and LXRα is associated with enhanced MTP expression and VLDL assembly/secretion (16). PGC-1β can interact with PPAR [symbol] (20) and activate the transcription of PPAR [symbol] target genes (21). Thus, the findings show that in the context of rat hepatoma cells, PGC-1β activates PPARα-RXRα (FIG. 8).

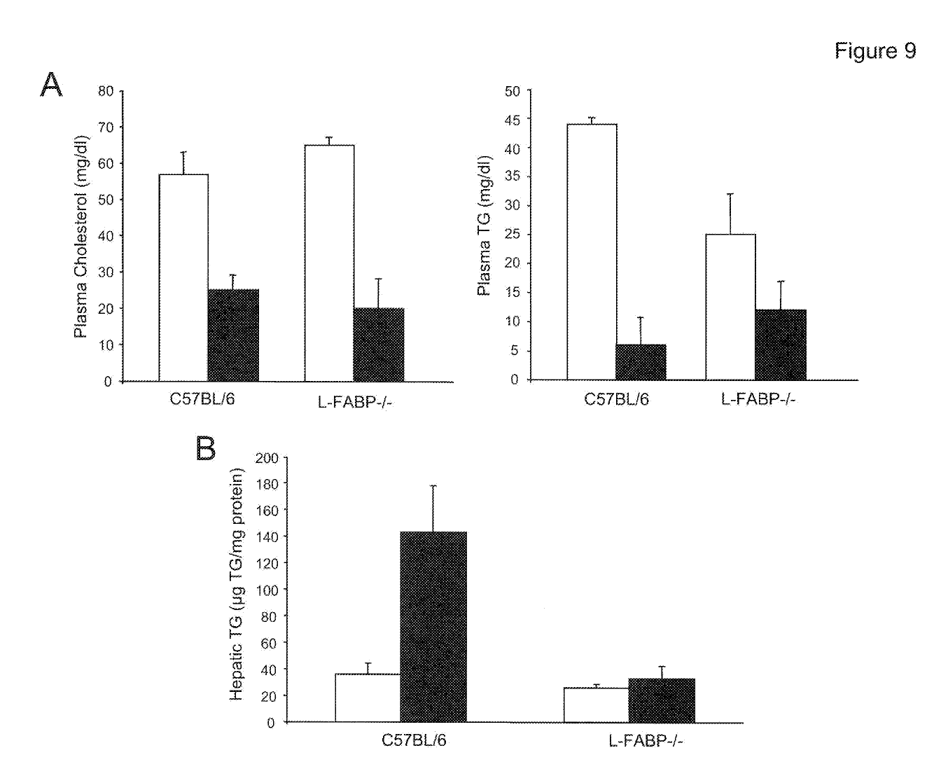

The combined data also suggest that retention of similar DR1 elements in the promoters of L-FABP and MTP ensures that their expression will be sufficiently induced to provide an efficient diversion of energy in the form of fatty acids to the VLDL assembly/secretion pathway. The same coordinate transcriptional regulation can attenuate the expression of both genes. The importance of coordinate decreased expression of L-FABP and MTP was clearly shown by additional studies showing that ablation of L-FABP blocked the accumulation of triglycerides in the livers of mice treated with the MTP inhibitor (FIG. 9). These findings have important implications regarding the efficacy of MTP inhibition as a target to ameliorate hyperlipidemia.

As previously indicated, MTP inhibition appears to be an effective therapeutic to reduce hyperlipidemia, but their use is associated with the development of fatty liver (22, 23). The data provided herein show that ablation of L-FABP completely blocks the accumulation of triglycerides in the liver of mice treated with the MTP inhibitor 8aR (FIG. 9B). These studies therefore show that agents that block either the function and/or expression of both L-FABP and MTP reduce hyperlipidemia without causing the development of fatty liver.

In one aspect of the invention, the disclosed methods and compositions can be used as part of a treatment regimen for hyperlipidemia. L-FABP and MTP cooperatively shunt fatty acids into glycerolipid synthesis and hepatic Very Low Density Lipoprotein (VLDL) secretion. Hyperlipidemia is the presence of elevated or abnormal levels of lipids and/or lipoproteins in the blood. Lipids (fatty molecules) are transported in a protein capsule, and the density of the lipids and type of protein determines the fate of the particle and its influence on metabolism. Thus, the invention provides compositions and methods for ameliorating (e.g., blocking or inhibiting) hyperlipidemia. In one embodiment, the method for treating hyperlipidemia provided herein includes administering to subject, an inhibitor of MTP in combination with an inhibitor of L-FABP. The MTP and L-FABP inhibitors may be administered as separate agents, or as a conjugate comprising a MTP inhibitor and a L-FABP inhibitor which are covalently bound.

Genetic disruption of L-FABP expression impairs the ability of the liver to efficiently import and transfer fatty acids into several metabolic pathways including glycerolipid biosynthesis and mitochondrial oxidation. Deletion of L-FABP diverts fatty acids for use by extra-hepatic tissue. Fatty acids are readily taken up and utilized by skeletal muscle and heart for the production of energy and heat. Fatty acids are also taken up by adipose tissue where they can be stored as triglycerides. L-FABP inhibitors can reduce or inhibit the development of hepatic steatosis caused by MTP inhibitors. An exemplary L-FABP inhibitor is 3-(decyldimethylsilyl)-N-[2-(4-methylphenyl)-1-phenylethyl]propanamide (Sandoz compound 58-035).

### Pharmaceutical Compounds

One aspect of the invention relates to the use, identification, making and research of pharmaceutical compounds which have the ability to treat hyperlipidemia through the inhibition of MTP and L-FABP. As set forth below, these compounds may take on many different formulations.

In some aspects of the invention, the pharmaceutical compounds are formed by covalently linking (a) one or more MTP inhibitors, and (b) one or more L-FABP inhibitors. That is, the pharmaceutical compound may be formulated as a single molecule comprising a small molecule MTP inhibitor(s) and a small molecule L-FABP inhibitor, wherein the MTP inhibitor(s) and the L-FABP inhibitor(s) are linked to one another by covalent bonding. Such molecules are sometimes referred to in the art as a co-drug.

There are a number of advantages to formulating the pharmaceutical compound as a co-drug. One advantage is that a co-drug formulation allows the MTP and L-FABP inhibitors to be delivered to, and absorbed by, patients in a precise stoichiometric ratio. This is advantageous when considering that patients may vary in their individual response to MTP and L-FABP inhibitors. Thus, for example, if a patient's response to a MTP inhibitor is proportionately greater than the patient's response to a L-FABP inhibitor, the pharmaceutical composition may be formulated as a co-drug that is proportionately higher in the L-FABP inhibitor (e.g. a conjugate of MTP and L-FABP inhibitors in a ratio of 1:2 respectively). Alternatively, the pharmaceutical compound may be stoichiometrically higher in L-FABP relative to the corresponding MTP inhibitor.

Formulating the pharmaceutical compound using defined stoichiometric ratios of MTP and L-FABP inhibitors also allows the pharmaceutical compound to assume a variety of combinations of MTP and L-FABP inhibitors. For example, the pharmaceutical compound may be formulated by conjugating two different MTP inhibitors with a single L-FABP inhibitor. Such methods for formulating the pharmaceutical compound of the invention, are particularly suited to screening assays aimed at identifying potential pharmaceutical combinations for testing as a treatment for hyperlipidemia in vivo.

Another advantage of formulating the pharmaceutical composition as a co-drug is that it provides a means for identifying effective stoichiometric ratios of MTP and L-FABP inhibitors that are effective in treating hyperlipidemia. This is accomplished by formulating a test co-drug (i.e. covalently bonded MTP and L-FABP inhibitors) having a defined stoichiometric ratio of MTP and L-FABP inhibitors.

The invention may be practiced with any stoichiometric combination of MTP and L-FABP inhibitors which provides a pharmaceutical compound that is useful in the treatment of hyperlipidemia, or useful in screening assays for identifying candidate pharmaceutical compounds for treatment of hyperlipidemia. Thus, the invention contemplates a pharmaceutical compound comprising at least one MTP inhibitor, and at least one L-FABP inhibitor, wherein the MTP inhibitor and the L-FABP inhibitor are linked by covalent bonding.

### Selection of MTP and L-FABP Inhibitors

The pharmaceutical compound of the invention may be formulated using any combination of MTP and L-FABP inhibitors (i.e. agents or “pharmaceutical agents”). In preferred embodiments, such inhibitors are pharmaceutically acceptable and do not otherwise cause an undesirable physical response when administered to the body of a subject.

In some embodiments, the pharmaceutical compound is formulated using MTP and L-FABP inhibitors (i.e. agents) that are organic molecules, preferably small organic compounds (i.e., small molecules) having a molecular weight of more than 100 and less than about 2,500 daltons. Such inhibitors should comprise functional groups necessary for structural interaction with proteins, particularly hydrogen bonding, and typically include at least an amine, carbonyl, hydroxyl or carboxyl group, preferably at least two of the functional chemical groups. The pharmaceutical actives often comprise cyclical carbon or heterocyclic structures and/or aromatic or polyaromatic structures substituted with one or more of the above functional groups.

In terms of small molecule MTP inhibitors, the invention may be practiced using any small molecule that can safely inhibit MTP when administered to a subject. That is, the invention may be practiced using small molecule inhibitors which are pharmaceutically acceptable and do not otherwise cause an adverse physiological reaction when administered to a subject. Suitable MTP inhibitors for formulating the pharmaceutical compound include, but are not limited to, AEGR-733 (Aegerion) (formerly BMS201038), AS-1552133 (Astellas), BAY-139952 (Bayer) (Implitamide, AEGR-427), BAY-139953 (Bayer), BMS-197636 (Bristol Myers Squibb), BMS-200150 (Bristol Myers Squibb), BMS-212122 (Bristol Myers Squibb), CP-319340 (Pfizer), CP-346086 (Pfizer), CP-467688 (Pfizer), GW-328713 (Glaxo), JTT-130 (Japan Tobacco), LAB-687 (Novartis), R-103757/Mitratapide/Yarvitan (Janssen), MTP-1307 (Maruko Seiyaku), MTP-1403 (Maruko Seiyaku) MTP-3115 (Maruko Seiyaku) (DM Rx from early 1990s), MTP-3631 (Maruko Seiyaku) (DM Rx from early 1990s), SLx-4090 (Surface Logix), T-0126 (Tanabe), and combinations thereof. Other suitable inhibitors that may be used alone, in combination, or in combination with the MTP inhibitors listed above, include those disclosed in the following references, the disclosures of which are incorporated herein by reference: U.S. Pat. Nos. 5,712,279, 5,741,804, 5,968,950, 6,066,653, and 6,121,283; PCT International Patent Application publications WO 96/40640, WO 97/43257, WO 98/27979, WO 99/33800 and WO 00/05201; European patent application publications EP 584446 and BP 643,057; and Biol Pharm Bull. 2005 February; 28(2):247-52. J Toxicol Sci. 2007 May; 32(2); 161-77.

MTP inhibitors for formulating the compounds of the invention may also be obtained using MTP inhibitor screening assays. In such embodiments, a test pharmaceutical agent (e.g. molecule) is measured for its ability to inhibit MTP using an in vitro and/or an in vivo assay. Agents that demonstrate MTP inhibitory activity are then identified as suitable MTP inhibitors for formulating the pharmaceutical compounds of the invention. In vitro and in vivo assays for identifying suitable MTP inhibitors include: incubating purified MTP or cellular homogenates with donor vesicles containing radiolabeled lipids (see e.g. J Lipid Res. 2004 April; 45(4):764-72. Epub 2004 Feb. 1); J Lipid Res. 2005 August; 46(8): 1779-85. Epub 2005 May 16; J Biol. Chem. 1995 Mar. 24; 270(12):6549-54 and measuring the rate of MTP-lipid transfer.

The pharmaceutical compound may be formulated using any small molecule that is capable of inhibiting L-FABP and which is pharmaceutically acceptable. One non-limiting example of a suitable L-FABP inhibitor is 3-(decyldimethylsilyl)-N-[2-(4-methylphenyl)-1-phenylethyl]propanamide (Sandoz compound 58-035) (lot #fr. 09061988), In some embodiments, such L-FABP inhibitors are identified using a screening assay. For example, a selected test agent is assayed for its ability to inhibit L-FABP in vitro. Suitable in vitro assays for testing L-FABP include, but are not limited to a high throughput L-FABP assay based on the 1,8-ANS binding assay (see: Kane, C. D., and Bernlohr, D. A. 1996, A simple assay for intracellular lipid-binding proteins using displacement of 1-anilinonaphthalene 8-sulfonic acid. Anal Biochem 233:197-204.) In this assay, recombinant L-FABP is incubated with a fluorescent surrogate ligand, 1-anilinonapthalene 8-sulfonic acid (ANS) and allowed to come to equilibrium. 1,8-ANS is non-fluorescent in solution but is highly fluorescent when bound in the hydrophobic, lipid-binding pocket of the fatty acid binding proteins. Experimentally, the simple benchtop laboratory assay was restructured for robotic manipulation in a high throughput format such that 1,8-ANS was added to the FABP in a 384-well format at its K_{d} value (predetermined from direct binding isotherms to be 1.5 μM) to generate a protein with half the sites bound. Chemical compounds in pools of 5 were added to each well and the fluorescence re-measured. For those compound pools where displacement is greater than 50%, secondary screens with individual molecules from each pool will be carried out. In tertiary screens, the primary targets will be titrated into the FABP bound with 1,8-ANS and the displacement quantitated as a function of ligand concentration.

The ability of an inhibitor of L-FABP, when administered, in combination with an inhibitor of MTP, to reduce plasma lipids without causing the development of hepatic steatosis can be determined by comparing the effect of the combined treatment on plasma lipids and hepatic lipids to those caused by treatment with an MTP inhibitor alone.

Without being limited to any particular theory, the pharmaceutical compounds of the invention may inhibit MTP and/or L-FABP activity by specific inhibition (e.g. competitive and non-competitive binding to the active site of MTP and/or L-FABP synthesis). It is also contemplated that the pharmaceutical compounds of the invention may inhibit the synthesis, including the post-translational processing of, MTP and L-FABP.

### Covalent Bonding

One aspect of the pharmaceutical compound concerns the covalent bonding that is used to link the MTP and L-FABP inhibitors. As used herein, the terms “covalent linkage,” “covalent bond,” “covalent bonding” or “bond” refer to a covalent bond between two distinct molecules such that there is the formation of stable single compound entity that has a definable chemical composition and structure. In general terms, the pharmaceutical compound of the invention may be formulated using any covalent linkage that allows the pharmaceutical compound to be administered to a subject as a single chemical. Depending on the structure of this chemical its metabolism by the subject and its effects on the physiology of the subject may vary. Therefore, the covalent linkage may be cleaved by processes inherent to the physiology of the subject. The tissue site of cleavage of the compound may affect the activity and site of action of the MTP and L-FABP inhibitors.

The covalent bond for linking the MTP and L-FABP inhibitors may be empirically designed to achieve a variety of physiological activities in the body of a subject. For example, the covalent bond, may assume a form that allows the pharmaceutical compound to be absorbed by the intestine intact, while being selectively cleaved once taken up by the liver. Identifying and providing a dual L-FABP/MTP that is more active in liver and less active in intestine would have the therapeutic advantage of reducing hepatic lipid secretion while avoiding the established side-effects associated with inhibiting intestinal lipid absorption (i.e. steatorrhea, the mal-absorption of essential fat soluble nutrients) yet achieve physiological activation in the liver.

The covalent bonding for linking the MTP and L-FABP inhibitors may take on a variety of chemical forms including, but not limited to, carboxylic esters, phospho-esters, amides (—R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof. The pharmaceutical compounds of the invention may impart their inhibitory activity through, a variety of mechanisms.

The invention provides methods for making the pharmaceutical compounds of the invention. One non-limiting example of such a method comprises the steps of (a) identifying at least one compound (i.e. active) that inhibits MTP, (b) identifying at least one compound (i.e. active) that inhibits L-FABP, and (c) bonding, such as by covalent bonding, the compound that inhibits MTP and the compound that inhibits L-FABP. The invention further contemplates pharmaceutical compositions made according to this method. One skilled in the art will appreciate the different experiments available for identifying MTP inhibitors and L-FABP inhibitors. Some non-limiting examples of such screening assays include, but are not limited to, those described above.

The invention also provides methods for making pharmaceutical compounds that can be screened for their ability to treat hyperlipidemia. One non-limiting example of such a method may be practiced by (a) identifying at least one agent that inhibits MTP, (b) identifying at least one agent that inhibits L-FABP, (c) bonding, such, as by covalent bonding, the agent(s) that inhibits MTP and the agent(s) that inhibits L-FABP thereby forming the pharmaceutical compound, and (d) testing the ability of the pharmaceutical compound to treat hyperlipidemia in vivo. The invention further contemplates pharmaceutical compounds made according to this method. In vivo methods for testing the efficacy of pharmaceutical compounds for treating hyperlipidemia are available in the art, such methods being taught in the following references, the disclosures of which are incorporated herein by reference: Spann, N. J., Kang, S., Li, A. C., Chen, A. Z., Newberry, E. P., Davidson, N. O., Hui, S. T., and Davis, R. A. 2006. Coordinate transcriptional repression of liver fatty acid-binding protein and microsomal triglyceride transfer protein blocks hepatic very low density lipoprotein secretion without hepatosteatosis. J Biol Chem 281:33066-33077.

As used in the present disclosure, “identifying” refers to the association of a pharmaceutical compound or agent with a desired activity (e.g. MTP or L-FABP inhibition, either in vivo or in vitro). Such identifying can occur through the in vivo or in vitro analysis (i.e. screening) of a test, agent for a desired activity. Identifying an agent having a desired activity may also occur through the association of compound with its activity which may be generally available in the art. Thus, an MTP or L-FABP inhibitor may be “identified” through an in vitro or in vivo assay, or through the association of agent's inhibitory activity based on knowledge generally available, in the art such knowledge attained from scientific publications.

Also contemplated are methods for using the pharmaceutical compounds of the invention in the treatment of hyperlipidemia. In general terms, and without being limited to any particularly theory, these methods are practiced by administering a therapeutically effective amount of a pharmaceutical compound that comprises at least one MTP inhibitor that is covalently linked (i.e. bonded) to at least one L-FABP inhibitor. In one non-limiting example of the invention, hyperlipidemia is treated by administering a pharmaceutical compound that comprises 8aR, and 3-(decyldimethylsilyl)-N-[2-(4-methylphenyl)-1-phenylethyl]propanamide (Sandoz compound 58-035) (lot #fr. 09061988), wherein 8aR and the Sandoz compound 58-035 are covalently linked.

Although pharmaceutical compounds comprising covalently linked MTP and L-FABP inhibitors are disclosed in detail, the invention also contemplates agents (e.g. non-conjugated small molecules) that have dual MTP and L-FABP inhibitor activity. Thus, the presently disclosed methods of making, using and screening for the pharmaceutical composition of the invention may also be practiced with iron-conjugated small molecules that have dual MTP and L-FABP inhibitor activity. It is further contemplated that the pharmaceutical compounds may comprise conjugated and non-conjugated agents selected from biomolecules including peptides, saccharides, fatty acids, steroids, purines, pyrimidines, derivatives, structural analogs or combinations thereof. It is also contemplated that these agents may be combined with, small molecule MTP and L-FABP inhibitors in conjugated or non-conjugated form.

### Methods of Use

In one embodiment, the method for treating hyperlipidemia includes administering to the subject a therapeutically effective amount of a nucleic acid molecule, such as double-stranded RNA (dsRNA), in order to induce RNA interference (RNAi) and silence MTP and/or L-FABP activity. RNAi is a phenomenon in which the introduction of dsRNA into a diverse range of organisms and cell types causes degradation of the complementary mRNA. In the cell, long dsRNAs are cleaved into short (e.g., 21-25 nucleotide) small interfering RNAs (siRNAs), by a ribonuclease. The siRNAs subsequently assemble with protein components into an RNA-induced silencing complex (RISC), unwinding in the process. The activated RISC then binds to complementary transcripts by base pairing interactions between the siRNA antisense strand and the mRNA. The bound mRNA is then cleaved and sequence specific degradation of mRNA results in gene silencing. As used herein, “silencing” refers to a mechanism by which cells shut down large sections of chromosomal DNA resulting in suppressing the expression of a particular gene. The RNAi machinery appears to have evolved to protect the genome from endogenous transposable elements and from viral infections. Thus, RNAi can be induced by introducing nucleic acid molecules complementary to the target mRNA to be degraded, as described in the examples below.

In another embodiment, the present invention provides a method of ameliorating or treating hyperlipidemia in a subject with the subject inhibitors. When used in the context of hyperlipidemia, the term “ameliorating” or “treating” means that the clinical signs and/or the symptoms associated with hyperlipidemia are lessened as a result of the actions performed. The signs or symptoms to be monitored will be characteristic of hyperlipidemia and will be well known to the skilled clinician, as will the methods for monitoring the signs and conditions. For example, chemical measures of lipid concentration have long been the most-used clinical measurement, not because they have the best correlation with individual outcome, but because these lab methods are less expensive and more widely available. However, there is increasing evidence and recognition of the value of more sophisticated measurements. Specifically, LDL particle number (concentration), and to a lesser extent size, have shown much tighter correlation with atherosclerotic progression and cardiovascular events than is obtained using chemical measures of total LDL concentration contained within the particles. LDL cholesterol concentration can be low, yet LDL particle number high and cardiovascular events rates are high.

In another aspect of the invention, the subject methods can be used as part of a treatment regimen for any indication that may result in hepatic steatosis, including but not limited to, viral infection and cancer. The term “cancer” as used herein, includes any malignant tumor including, but not limited to, carcinoma, sarcoma. As such, the compositions and methods of the invention may be used for the treatment of any cancer characterized by elevated MTP and L-FABP activity, such as hepatoma. As used herein, “hepatoma” refers to carcinoma of the liver. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. In some cases, the treatment of cancer may include the treatment of solid tumors or the treatment of metastasis. Metastasis is a form of cancer wherein the transformed or malignant cells are traveling and spreading the cancer from one site to another.

The terra “cancerous cell” as used herein. Includes a cell afflicted by any one of the cancerous conditions provided herein. Thus, the methods of the present invention include treatment of benign overgrowth of melanocytes, glia, prostate hyperplasia, and polycystic kidney disease. The term “carcinoma” refers to a malignant new growth made up of epithelial cells tending to infiltrate surrounding tissues, and to give rise to metastases.

In certain embodiments, the invention compounds may further be administered in combination with an anti-inflammatory, antimicrobial, antihistamine, chemotherapeutic agent, antiangiogenic agent, immunomodulator, therapeutic antibody or a protein kinase inhibitor, e.g., a tyrosine kinase inhibitor, to a subject in need of such treatment. Other agents that may be administered in combination with invention compounds include protein therapeutic agents such as cytokines, immunomodulatory agents and antibodies. While not wanting to be limiting, antimicrobial agents include antivirals, antibiotics, anti-fungals and anti-parasitics. When other therapeutic agents are employed in combination with the compounds of the present invention they may be used for example in amounts as noted in the Physician Desk Reference (PDR) or as otherwise determined, by one having ordinary skill in the art.

In another aspect, the invention provides a method to ameliorate viral infections, such as hepatitis C virus (HCV) and to reduce the development of inflammatory liver disease, fibrosis, cirrhosis, hepatic failure, and/or hepatocellular carcinoma. The processes responsible for the production and transport of HCV particles share many aspects associated with the production and transport of plasma lipoproteins. HCV forms a lipoprotein complex and is transported in plasma as a component of both VLDL and LDL (Andre, et al., 2005, Hepatitis C virus particles and lipoprotein, metabolism. Semin Liver Dis 25:93-104). Hepatic production of HCV impairs the expression and activity of MTP, as well as the secretion of VLDL (Domitrovich, et al., 2005, Hepatitis C virus nonstructural proteins inhibit apolipoprotein B100 secretion. J Biol Chem 280:39802-39808). Inhibition of MTP may explain why patients infected with HCV exhibit altered plasma lipid levels (Siagris, et al., 2006, Serum lipid pattern in chronic hepatitis C: histological and virological correlations. J Viral Hepat 13:56-61) and their livers develop steatosis (Mirandola, et al, 2006, Liver microsomal triglyceride transfer protein is involved in hepatitis C liver steatosis. Gastroenterology 130:1661-1669). Conversely, MTP inhibitors block the production of HCV by hepatoma cells (Huang, et al., 2007, Hepatitis C virus production by human hepatocytes dependent on assembly and secretion of very low-density lipoproteins. Proc Natl Acad Sci USA 104:5848-5853). The invention describes a method to inhibit MTP (and thus the production of HCV) without causing the development of steatosis. Thus, co-administration of L-FABP inhibitors with MTP inhibitors to patients infected with HCV will reduce the production of HCV and ameleorate the pathologies associated with chronic HCV infection, the development of steatosis, cirrhosis and hepatic failure (Hwang, et al., 2001, Hepatic steatosis in chronic hepatitis C virus infection: prevalence and clinical correlation. J Gastroenterol Hepatol 16:190-195).

In another aspect of the invention, a method for identifying an agent useful for hyperlipidemia is provided. An agent useful in any of the methods of the invention can be any type of molecule, for example, a polynucleotide, a peptide, a peptidomimetic, peptoids such as vinylogous peptoids, a small organic molecule, or the like, and can act in any of various ways to further reduce or inhibit MTP and L-FABP expression or activity. The agent can be administered in any way typical of an agent used to treat the particular type of hyperlipidemia, or under conditions that facilitate contact of the agent with the target cells and, if appropriate, entry into the cells. Entry of a polynucleotide agent into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. Generally, an agent is formulated in a composition (e.g., a pharmaceutical, composition) suitable for administration to the subject. Such formulated agents are useful as medicaments for treating a subject suffering from hyperlipidemia that is characterized, in part, by elevated MTP and L-FABP activity or expression.

Candidate agents encompass numerous chemical classes, though typically they are organic molecules, preferably small organic compounds (i.e., small molecules) having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In another aspect, the methods of the invention are useful for providing a means for practicing personalized medicine, wherein treatment is tailored to a subject based on the particular characteristics of the hyperlipidemia in the subject. The method can be practiced, for example, by contacting a sample of cells from the subject with at least one test agent or MTP and L-FABP inhibitor, wherein a decrease in MTP and L-FABP activity or expression in the presence of the test agent or inhibitor as compared to the MTP and L-FABP activity or expression in the absence of the test agent or inhibitor identifies the agent or inhibitor as useful for treating the disease. The sample of cells examined according to the present method can be obtained from the subject to be treated, (a) cells of an established cell line, or (b) cells from a subject having the same type of hyperlipidemia as that of the subject. In one aspect, the established cell line can be one of a panel of such cell lines, wherein the panel can include different cell lines of the same type of disease and/or different cell lines of different diseases associated with elevated levels of MTP and L-FABP activity or expression. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease is established and a treatment protocol is initiated, the methods of the invention may be repeated on a regular basis to evaluate whether the level of MTP and L-FABP activity of expression in the subject begins to approximate that which is observed in a normal subject. The results obtained from successive assays may be used to show the efficacy of treatment over a period ranging from several days to months. Accordingly, the invention is also directed to methods for monitoring a therapeutic regimen for treating a subject having hyperlipidemia. A comparison of the level of MTP and L-FABP activity or expression prior to and during therapy indicates the efficacy of the therapy. Therefore, one skilled in the art will be able to recognize and adjust the therapeutic approach as needed.

All methods may further include the step of bringing the active ingredient(s) (e.g. pharmaceutical compound) into association with a pharmaceutically acceptable carrier, which constitutes one or more accessory ingredients. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The route of administration of a composition containing the inhibitors of the invention will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the inhibitor is a small organic molecule such as a steroidal alkaloid, it cars be administered in a form that releases the active agent at the desired position in the body (e.g., the liver), or by injection into a blood vessel such that the inhibitor circulates to the target cells (e.g., hepatoma cells).

Exemplary routes of administration include, but are not limited to, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of a tumor, for example, intravenously or intra-arterially into a blood vessel supplying the tumor.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art, would know that the amount of the inhibitor of MTP and L-FABP activity or expression to treat hyperlipidemia or hepatoma in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

The methods of the invention can be performed by contacting samples of cells ex vivo, for example, in a culture medium or on a solid support. An advantage of the in vivo assay is that the effectiveness of a test agent can be evaluated in a living animal, thus more closely mimicking the clinical situation. Since in vivo assays generally are more expensive, they can be particularly useful as a secondary screen, following the identification of “lead” agents using an in vitro method.

When practiced as an in vitro assay, the methods can be adapted to a high throughput format, thus allowing the examination of a plurality (i.e., 2, 3, 4, or more) of cell samples and/or test agents, which independently can be the same or different, in parallel. A high throughput format provides numerous advantages, including that test agents can be tested on several samples of cells from a single subject, thus allowing, for example, for the identification of a particularly effective concentration of an agent to be administered to the subject, or for the identification of a particularly effective agent to be administered to the subject. As such, a high throughput format allows for the examination of two, three, four, etc., different test agents, alone or in combination, on the cancer cells of a subject such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test agent(s); or two or more samples of cells obtained from one subject can be examined with same or different amounts of one or a plurality of test agent. In addition, cell samples, which can be of the same or different subjects, can be examined using combinations of test agents and/or known effective agents. Variations of these exemplified formats also can be used to identifying an agent or combination of agents useful for treating cancers.

When performed in a high throughput, (or ultra-high throughput) format, the methods can be performed on a solid support (e.g., a microliter plate, a silicon wafer, or a glass slide), wherein samples to be contacted with a test agent are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for decreased MTP and L-FABP activity or expression.

Positive controls and negative controls may be used in the assays of the invention. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc., which may be used to facilitate optimal protein-protein binding and/or reduce non-specific or background interactions. Also reagents that otherwise improve the efficiency of the assay, such as protease, inhibitors, nuclease inhibitors, anti-microbial agents, etc., may be used. The mixture of components may be added in any order that provides for the requisite binding.

The measurements can be determined wherein all of the conditions are the same for each measurement, or under various conditions, with or without test agents, or at different stages of a disease state such as cancer. For example, a measurement can be determined in a cell or cell population wherein a test agent is present and wherein the test agent is absent. In another example, the cells may be evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In yet another example, the measurements of Dkk activity are token wherein the conditions are the same, and the alterations are between one cell or cell population and another cell or cell population.

In another aspect, the invention provides kits for performing the methods of the invention that include one or more inhibitors of MTP and/or L-FABP activity or expression. In one embodiment, the invention provides kits that includes a pharmaceutical composition comprising one or more inhibitors of MTP and/or L-FABP activity or expression. The included inhibitors may be a dsRNA that hybridizes to a polynucleotide encoding or regulating MTP or a functional fragment thereof, and a dsRNA that hybridizes to a polynucleotide encoding or regulating L-FABP or a functional fragment thereof. In another embodiment, the kit includes instructions for practicing the methods of the invention.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Deleting L-FABP Expression Prevents the Development of Hepatic Steatosis

This example demonstrates use of L-FABP inhibitors as a means to prevent the development of hepatic steatosis caused by MTP inhibitors.

Cell culture. Cells were cultured and transfected as described (4). FAO cells were obtained as a gift from the University of Colorado. L35 cells were obtained as described (24).

Cells were transfected using LipofectAMINE reagent (Invitrogen) according to manufacturer's protocol, with minor modifications (4). One day prior to transfection, L35 and FAO cells (2×10⁵) were seeded on 12-well plates. On day of transfection, cells were transfected 0.8 μg of promoter/luciferase reporter construct and 6 ng of pRL-CMV plasmid as an internal control for normalization of L-FABP and MTP promoter activities. The normalized pRL-CMV activities are reported relative to activity of the empty vector from parallel experiments. Varying doses of COUP-TFII expression vector was added as indicated in figure legends. The total DNA concentration for each assay was maintained constant by addition of empty expression vector pCR 3.1 (Invitrogen). Upon transfection, cells were incubated for 48 h and harvested using passive lysis buffer (Promega). Luciferase activities were measured using the Dual-Luciferase Reporter Assay system (Promega).

The L-FABP and MTP promoter reporter assays using the PPARα and RXR agonists, WY-14,643 (WY) and 9-cis retinoic acid (cRA) respectively (A.G. Scientific, Inc.), were performed as described in figure legends. Both WY-14,643 and 9-cis retinoic acid were dissolved in dimethyl sulfoxide (DMSO, 0.15% v/v) and used at working concentrations of 10 μM (WY) and 1 μM (cRA). Briefly, upon transfection cells were treated for 48 h with agonists or DMSO alone as indicated. Cells were harvested and promoter activity assayed as described above.

Reporter Gene Constructs and Expression Vectors. The wild type and mutant rat MTP reporter vectors (−135/+66) were as described previously (4). To generate the wild type rat L-FABP reporter vector (−141/+66), genomic DNA was isolated and purified from FAO cells using the DNeasy tissue kit (Qiagen). The promoter fragment was generated by PCR using the primers with indicated restriction enzyme sites, forward 5′(KpnI)-GAACAAACTTCTGCCGGTACCATTCTGATTTTTA-3′ (SEQ ID NO: 1) and reverse 5′(BglII)-TTCATGGTGGCAATGAGATCTCCTTTCCACAGCTGA-3′ (SEQ ID NO: 2). The promoter fragment was then cloned into KpnI and BglII sites of the empty luciferase reporter vector PGL3Basic (Promega).

To generate the mutant L-FABP reporter vector a specific mutation in the proximal DR1 sequence was generated using the QuikChange site-directed mutagenesis kit (Stratagene). In vitro mutagenesis the rat L-FABP (−141/+66)-luciferase reporter vector as the template and two oligonucleotide primers (mutated bases underlined), each complementary to opposite strands of the vector (forward, 5′-AATCGACAATCACTGTGCTATGGCCTATATTT-3′ (SEQ ID NO: 3); reverse, 5′-AAATATAGGCCATAGCACAGTGATTGTCGATT-3′) (SEQ ID NO; 4). The site-specific mutant construct was verified by DNA sequencing. The expression plasmid for COUP-TFII was a gift from Baylor Medical University.

Preparation of Nuclear Extracts. Nuclear extracts from L35 and FAO cells were prepared as described previously (Kang, 2003). Briefly, cells were trypsinized and harvested by centrifugation, washed with 1× phosphate-buffered saline, and resuspended in a hypotonic buffer (10 mM HEPES, pH7.9 at 4° C., 25% glycerol, 1.5 mM MgCl2, 10 mM KCl, 0.2 mM phenylmethylsulfonylfluoride, 0.5 mM dithiothreitol). After a 10-min incubation on ice, cells were lysed with use of a Dounce homogenizer. The nuclei were pelleted by centrifugation and resuspended in low salt buffer (20 mM HEPES, pH7.9 at 4° C., 25% glycerol, 1.5 mM MgCl2, 0.02 mM KCl, 0.2 mM EDTA, 0.2 mM phenylmethylsulfonylfluoride, 0.5 mM dithiothreitol). Subsequently, the high salt buffer (20 mM HEPES, pH7.9 at 4° C., 25% glycerol, 1.5 mM MgCl2, 1.2 mM KCl, 0.2 mM EDTA, 0.2 mM phenylmethylsulfonylfluoride, 0.5 mM dithiothreitol) was added dropwise with stirring. The resulting suspension was rocked gently for 30 min to allow extraction of nuclear proteins. The nuclei were centrifuged again for 30 min and the resulting supernatant was dialyzed for 1 h against, dialysis buffer (20 mM HEPES, pH7.9 at 4° C., 20% glycerol, 100 mM KCl, 0.2 mM EDTA, 0.2 mM phenylmethylsulfonylfluoride, 0.5 mM dithiothreitol).

Electrophoretic Mobility Shift Assays. All oligonucleotides used for EMSAs were synthesized by IDT. The following oligonucleotides (sense strands) were used in gel mobility shift assays: MTP-DR1, 5′-TGACCTTTCCCCTATAGATAAACACTGTTG-3′ (SEQ ID NO: 5); mutant MTP-DR1, 5′-TGTGCTTTCCCCTATAGATAAACACTGTTG-3′ (SEQ ID NO: 6); L-FABP-DR1, 5′-TGACCTATGGCCTATATTTGAGGAGGAAGA-3′ (SEQ ID NO: 7); mutant L-FABP-DR1, 5′-TGTGCTATGGCCTATATTTGAGGAGGAAGA-3′ (SEQ ID NO: 8).

The probes were prepared by annealing the complementary oligonucleotides and by end labeling with [γ-³²P]ATP (3000 mCi/mmol), PerkinElmer Life Sciences) using T4 polynucleotide kinase (New England Biolabs), followed by purification on a G50 column. For binding reactions, 15 μg of nuclear extracts were incubated with 3×104 cpm probe on ice for 20 min in a total volume of 15 μl of solution (20 mM HEPES, pH7.9 at 4° C., 10% glycerol, 100 mM KCl, 1 mM EDTA, and 2 μg poly(dI-dC). For supershift experiments, 1 μl of specific antibodies were added to preincubated DNA-protein complexes for an additional 20 min on ice. Antibodies against COUP-TFII (sc-6576X), RXRα (sc-553X), and PPARα (sc-9000X) were obtained from Santa Cruz Biotechnology, Inc. DNA-protein complexes were resolved on 4% native polyacrylamide gel electrophoresis containing 0.5×TBE buffer.

cDNA synthesis and Real-time PCR. Total RNA was isolated from either frozen liver using the Versagene RNA Tissue Kit (Gentra Systems, Inc.) or from cells using the Versagene RNA Cell Culture Kit (Genera Systems, Inc.) with on-column DNA removal per manufacturer's instructions. The RNA concentrations were determined by spectrophotometer at 260 nm. First strand cDNA was synthesized from 0.5 μg of total RNA using the BioRad iScript for reverse transcription (BioRad). Specific primers for each gene (Supplementary Table 1) were designed using gene sequences from GenBank™. To avoid amplification of genomic DNA, the primers were positioned to span exon junctions. All primers were synthesized by IDT.

Real-time PCR analysis was performed with the BioRad iCycler using BioRad SyBr Green supermix according to manufacturers instruction. The reactions were analyzed in triplicate with specific product, monitored using melt-curve analysis. The expression data were normalized to an endogenous control, either 18S ribosomal RNA or acidic ribosomal phosphoprotein P0 (36B4). The level of both 18S RNA and 36B4 was invariable among samples of all experiments. The relative expression levels were calculated according to the formula 2^{-ΔCt}, where ΔCt is the difference in threshold cycle (Ct) values between the target and either the 18S or 36B4 endogenous control.

Chromatin Immunoprecipitation Assay and Relative Quantitation. Cells were cultured in complete medium in 150-mm dishes until ≈70-80% confluent. Where indicated the agonists WY (10 μM) and cRA (1 μM) were added to cell culture medium for 48 h prior to harvesting. The cells were then fixed by the addition of 280 μl of 37% formaldehyde (Sigma) to 10 ml of culture medium for 10 min at 37° C., harvested, and processed for immunoprecipitation using the ChIP-IT Shearing Kit (Active Motif) and ChIP-IT Chromatin Immunoprecipitation Kit (Active Motif) for chromatin immunoprecipitation according to manufacturer's protocol. Immune complexes were eluted, reverse cross-linked using 5M NaCl at 65° C., treated with proteinase K, and purified using mini-columns provided with ChIP-IT kit.

Specific genomic DNA fragments from immunoprecipitated samples and inputs were quantitated by Real-time PCR with BioRad SyBr Green Supermix as indicated above. As a control for region selectivity of immunoprecipitation-specific enrichment differences, amounts of non-coding distal untranslated regions were determined for each sample. The antibodies used were against COUP-TFII (Wyeth-Ayerst Research, PA), PPARα (sc-9000X, Santa Cruz Biotechnology, Inc.) and control IgG (Active Motif). Primer sets were designed to amplify the following rat genomic DNA regions: MTP-DR1 (forward-5′-TAGTGAGCCCTTCCATGAAC-3′ (SEQ ID NO: 9); reverse-5′-CAGAATCTGCGACAACAGTG-3′) (SEQ ID NO: 10), L-FABP-DR1 (forward-5′-GAGTTAATGTTTGATCCTGGCC-3′ (SEQ ID NO: 11); reverse-5′-CCACCCACTGTTGGCTATTTT-3′) (SEQ ID NO: 12), and L-FABP-3-untranslated region (forward-5′-GTCTTCCGCTACCTAAGAGG-3′ (SEQ ID NO: 13); reverse-5′-CTGTCATCTGACCAGCTCTC-3′) (SEQ ID NO: 14). All values were normalized to values from both input DNA and immunoprecipitations with IgG using the ΔΔCt method. Briefly, for every promoter studied a ΔCt value was calculated for each sample by subtracting the Ct value for the input DNA from the Ct value obtained for the immunoprecipitated sample, A ΔΔCt value was then calculated by subtracting the ΔCt value for the sample immunoprecipitated with the specific antibody (PPAR [symbol] or COUP-TFII) from the ΔCt value for the corresponding sample immunoprecipitated with normal rabbit serum (IgG). Fold differences (factor-specific ChIP relative to control IgG ChIP) were then determined by raising 2 to the ΔΔCt power.

RNA interference. Knockdown of PPARα in FAO cells was achieved by using the Smartpool siRNA (Dharmacon) specific for rat PPARα. The sequence for siRNA directed against rat PGC-1β was selected as described previously (16). The rat PGC-1β siRNA (sense sequence; 5′-GATATCCTCTGTGATGTTA-3′) (SEQ ID NO: 15) was synthesized by Dharmacon as a 21-nucleotide duplex, using option A4, with 3′ dinucleotide (TT) overhangs. The species-specific siCONTROL non-targeting siRNA #1 (Dharmacon) was utilized as a negative control sequence to monitor non-specific targeting. Cells were plated in 12-well plates at a concentration of 5×10⁴/well 24 h prior to experiment. Prior to transfection, siRNAs were resuspended in 1×siRNA Buffer (Dharmacon) to a concentration of 20 μM. All siRNAs were transfected into FAO cells using the DharmaFECT™ 4 transfection reagent (Dharmacon) according to manufacturer's instructions. FAO cells were transfected with the indicated siRNAs for 48-72 h at working concentrations of 100 nM as indicated in figure legends. For the knockdown of PGC-1β, cells were transfected with the indicated siRNAs for 72 hours; cells were exposed to the agonists WY-14,643 (10 μM) and 9-cis RA (1 μM) 24 hours post-transfection and treated for 48 hours prior to harvesting. RNA isolation, cDNA synthesis and Real Time PCR expression analyses were performed as described above.

Animal Studies. Male PPARα−/− mice and age matched WT littermates (on SV/129 background) were fed a standard chow diet, supplemented with either the PPARα agonist GW-7647 (2.5 mg/kg/d) or equivalent amount of solvent vehicle (DMSO), for 7 weeks. All animals had ad libitum access to water. Mice were weighed every 2 weeks and drug intake was adjusted according to mean weight. Upon end of treatment animals were sacrificed, liver was isolated, and total RNA was extracted using Versagene RNA Tissue Kit (Gentra Systems, Inc.). First-Strand cDNA synthesis and Real Time PCR expression analyses were performed as described above.

For MTP Inhibitor (8aR) studies male L-FABP−/− mice and age matched WT controls (C57BL/6) were fed a standard chow diet, then administered the 8aR compound orally each day for 7 days at a dose of 50 mg/kg body weight. Upon end of treatment animals were sacrificed, plasma and livers were isolated, and the various hepatic and plasma lipid levels were determined using commercially available kits as described previously (25).

Lipid Extraction and Analysis. Livers were homogenized in PBS and protein concentration determined. 300 μl of homogenate was extracted with 5 ml of chloroform methanol (2:1) and 0.5 ml 0.1% sulfuric acid. An aliquot of the organic phase was collected, dried under nitrogen, and resuspended in 2% Triton X-100, Hepatic FFA, TG, and cholesterol content were determined using commercially available kits as described previously (25). Data were normalized for differences in protein concentration.

Adenovirus Experiment Adenoviral vectors expressing either PGC-1 [symbol] Ad-PGC-1β) or GFP (Ad-GFP) were generous gifts from Dana-Farber Cancer Institute, Harvard Medical School, Boston, Mass., L35 cells were infected with either the Ad-PGC-1β or Ad-GFP for 2 hours in serum-free media, then treated for 48 hours with the complete media with the agonists WY-14,643 (10 μM) and 9-cis RA (1 μM). Cells were infected ˜75-85% as determined by GFP expression. RNA isolation, cDNA synthesis and Real Time PCR expression analyses were performed as described above.

Immunoprecipitation of secreted apoB. Secreted apoB was immunoprecipitated as previously described (3, 26). Briefly, L35 cells were cultured in 60 mm dishes in the absence or presence of WY-14,643 (10 μM) and 9-cis RA (1 μM) for 72 hrs. Cells were then switched to methionine-free DMEM for 2 hrs and then labeled with 3 ml [³⁵S]-methionine (100 μCi/ml) in DMEM for 24 hrs. Media were collected and incubated with polyclonal anti-apoB antibody overnight at 4° C. Protein A-sepharose was then added and the mixture was further incubated for 2 hrs at 4° C. The immunoprecipitate complex was washed 3 times with TETN buffer (25 mM Tris at pH 7.5, 5 mM EDTA, 250 mM NaCl and 1% Triton X-100) and once with PBS. The pellet was resuspended in SDS-PAGE loading buffer, boiled for 5 min and resolved on a 4-12% Tris-glycine gel by electrophoresis. Radioactive proteins were detected by autoradiography.

Transcriptional activities of MTP and L-FABP promoter-reporters reflect cell-type specific differences in mRNA expression. A DR1 element located within the proximal MTP promoter region was previously shown to be responsible for the lack of expression in L35 cells and the high level expression exhibited by FAO cells (4). Occupation of this DR1 element by COUP-TFII was shown responsible for repressed MTP gene transcription exhibited by L35 cells (4). The L-FABP gene, whose product is involved in the regulation of VLDL assembly and secretion (25), was examined to determine whether it would be regulated by a similar mechanism. Sequence analysis of the L-FABP promoter (FIG. 1A) indicated that it contains a DR1 element which is similar to the DR1 element responsible for transcriptional regulation of the MTP gene (4). Moreover, expression of L-FABP mRNA by L35 and FAO cells paralleled the expression of MTP mRNA (FIG. 1B) suggesting that the transcription of both genes may be coordinately regulated.

Luciferase reporter constructs containing either the L-FABP- or MTP-proximal promoter regions displayed similar cell-type specific differences; promoter activities were approximately 8-fold higher in FAO cells relative to levels in L35 cells (FIG. 1C). Mutational deletion of the DR1 site, in either L-FABP or MTP promoter constructs, decreased the levels of promoter activity in FAD cells to levels similar to those seen in L35 cells (FIG. 1B). Thus, the proximal DR1 elements residing within the promoter regions of the L-FABP and MTP genes are sufficient to confer relative promoter activities which correlate with the endogenous mRNA levels of both genes displayed by the L35 and FAO cell lines.

Binding of COUP-TFII to the proximal DR1 site mediates transcriptional repression of the L-FABP gene. Nuclear extracts obtained from the L35 and FAO hepatoma cell lines formed distinct DNA-protein complexes with the oligonucleotide probe containing the DR1 element of the L-FABP promoter (FIG. 2A). The DNA-protein complex formed using nuclear extracts from L35 cells exhibited a supershift with an antibody specific for COUP-TFII (FIG. 2A). In contrast, the DNA-protein complex formed using nuclear extracts from FAO cells did not display a supershift with the COUP-TFII antibody (FIG. 2A). Since mutation of the DR1 site blocked the formation of the cell-type specific DNA-protein complexes, they required an intact DR1 site.

Ectopic expression of COUP-TFII in FAO cells transfected with a COUP-TFII expression plasmid resulted in a dose dependent decrease in L-FABP promoter activity (FIG. 2B). Since increased COUP-TFII expression did not alter the activity of an L-FABP promoter construct harboring a mutational deletion of the DR1 sequence (FIG. 2B), the repression of the L-FABP promoter by COUP-TFII is dependent on a functional DR1 element. The maximal reduction of transcription exhibited by FAO cells transfected with the plasmid expressing COUP-TFII only partially (50-70%) recapitulated the low level exhibited by L35 cells suggesting that additional factors are likely to contribute to the cell-type specific differences in expression. These Findings, similar to those obtained using the MTP DR1 element (4), support the conclusion that in L35 cells occupancy of these DR1 elements by COUP-TFII is responsible for transcriptional inactivation of both genes (FIG. 2A).

PPARα-RXRα heterodimers compete with COUP-TFII for binding to the DR1 promoter elements of both the MTP and L-FABP genes. EMSA-supershift analyses of complexes formed with the MTP-DR1 site revealed a FAO cell-specific complex containing RXR [symbol] (4). Since PPARα-RXR [symbol] heterodimers have been shown to activate the transcription of L-FABP via the DR1 site in the proximal promoter region (11), it was assessed whether the MTP gene was regulated in a similar manner. EMSA-supershift analyses using nuclear extracts from FAO cells demonstrated that DNA probes containing either the L-FABP- or MTP-DR1 sites formed similar FAO-specific complexes (FIG. 3A; lane 1), which did not supershift with a COUP-TFII specific antiserum (FIG. 3A; lane 2), but did supershift with an antiserum recognizing either RXRα (FIG. 3; lane 3) or PPARα (FIG. 3; lane 4). In contrast, PPARα-RXRα supershifts were not detected with the nuclear extracts obtained from L35 cells ((4)). These Findings indicate that in FAO cells, the DR1 sites of both the L-FABP and MTP promoters are occupied by PPARα-RXRα heterodimers while these same sites are occupied by a COUP-TFII complex in L35 cells.

Chromatin immunoprecipitation (ChIP) analyses using a COUP-TFII-specific antiserum showed that containing chromatin from L35 cells exhibited a 4-fold enrichment of L-FABP-DR1 and MTP-DR1 region, compared to chromatin cells from FAO cells (FIG. 3B). In contrast, using a PPARα-specific antiserum, chromatin obtained from FAO cells, exhibited a ˜4-fold enrichment of the proximal L-FABP-DR1 and MTP-DR1 regions compared to chromatin from L35 cells (FIG. 3B). Since the levels of distal untranslated regions immunoprecipitated from both cell lines were similar (FIG. 3B), the ˜4-fold enrichment of DNA sequences containing the proximal L-FABP-DR1 and MTP-DR1 regions reflect cell-type specific differences in binding of PPARα-RXRα (FAO cells) or COUP-TFII (L35 cells) to both the L-FABP and MTP promoters. Thus, the data obtained from the ChIP analyses (FIG. 3B) were concordant with the data obtained from the EMSA-supershift analyses (FIGS. 2A and 3A).

PPARα-RXR [symbol] agonist treatment of L35 cells results in the transcriptional induction of L-FABP and MTP expression and a restored ability to secrete apoB. Treatment of L35 cells with either a PPARα and/or an RXRα agonist markedly increased the expression of both MTP (˜55-65 fold) and L-FABP (˜60-75 fold) mRNAs (FIG. 4A). Treatment with both agonists synergistically increased MTP and L-FABP mRNA expression by nearly 300-fold (FIG. 4A). MTP and L-FABP promoter luciferase reporter constructs exhibited similar responses to the PPARα and RXRα agonists (FIG. 4B). Thus, treatment of L35 cells with PPARα and RXR [symbol] agonists (both separately and combined) enhanced the transcriptional activities of both the MTP- and L-FABP-DR1 promoter reporter constructs by up to 10-fold (FIG. 4B). No significant changes in the activities of either promoter were detected using reporter constructs harboring mutant DR1 sites (FIG. 4B). These findings indicate that the effects of PPARα and RXR [symbol] agonists are mediated through the DR1 elements.

It should be noted that treatment with the vehicle DMSO alone caused a relatively modest but significant increase in both the expression of MTP and L-FABP mRNA and the activities of their promoter-reporters (FIGS. 4A and 4B, respectively). Clearly, PPARα and RXR [symbol] agonists added to cells using DMSO as a vehicle exhibited a greater induction of L-FABP and MTP than did DMSO vehicle alone (FIGS. 4A and 4B). The induction of PPAR [symbol] activated genes by DMSO has been described (27).

L35 cells lack the ability to assemble and secrete apoB-containing lipoproteins due to transcriptional inactivation of the MTP gene (3, 4). Treating L35 cells with PPARα-RXR [symbol] agonists markedly enhanced the secretion of de novo synthesized ³⁵S-labeled apoB (FIG. 4C). Thus, PPARα-RXR [symbol] agonists restored expression of L-FABP and MTP, which lead to an activation of apoB-containing lipoprotein assembly and secretion in L35 cells.

Agonist induction of L-FABP and MTP are caused by changes of the relative cellular content of the DR1-associated factors and altered complex occupation of the proximal elements of both genes. Further analyses revealed that treating L35 cells with PPARα-RXRα agonists altered the levels of nuclear receptors so that they resembled the levels displayed by FAO cells (FIG. 5A). Thus, following treatment of L35 cells with PPARα-RXRα agonists expression of both PPARα (5-fold) and RXRα (2.3-fold) mRNAs were increased, whereas the level of COUP-TFII was decreased (−60%) (FIG. 5A). L35 cells treated with DMSO alone also exhibited a drastic reduction in COUP-TFII levels, but no change in the levels of either PPARα or RXRα (FIG. 5A). The DMSO-mediated reduction in COUP-TFII may explain why DMSO alone was associated with increased transcription and expression of L-FABP and MTP (FIG. 4).

To assess whether the agonist-mediated changes in expression of the transcriptional factors (FIG. 5A) coincided with an alteration in complex occupancy of the DR1 sites in the endogenous promoters, ChIP analyses of agonist-treated L35 cells were compared to that of untreated L35 and FAO cells. PPARα-RXRα agonist treatment of L35 cells decreased (˜3-fold) the amount of L-FABP- and MTP-DR1 region-containing chromatin immunoprecipitated with the COUP-TFII-specific antiserum (FIG. 5B), while it increased (˜3-fold) the amount bound by a PPARα-specific antiserum (FIG. 5B). Thus, the results from the ChIP analyses were concordant with the changes in expression levels of the nuclear receptors (FIG. 5A). The combined data indicate that PPARα-RXRα ligand activation converts the L35 cell type phenotype into the FAO cell type phenotype by increasing the cellular content of activator complex PPARα-RXRα relative to the repressor COUP-TFII. These changes result in parallel changes in the complex associating with the DR1 elements of both the L-FABP and MTP genes, favoring DR1 occupation by the activating PPARα-RXRα complex.

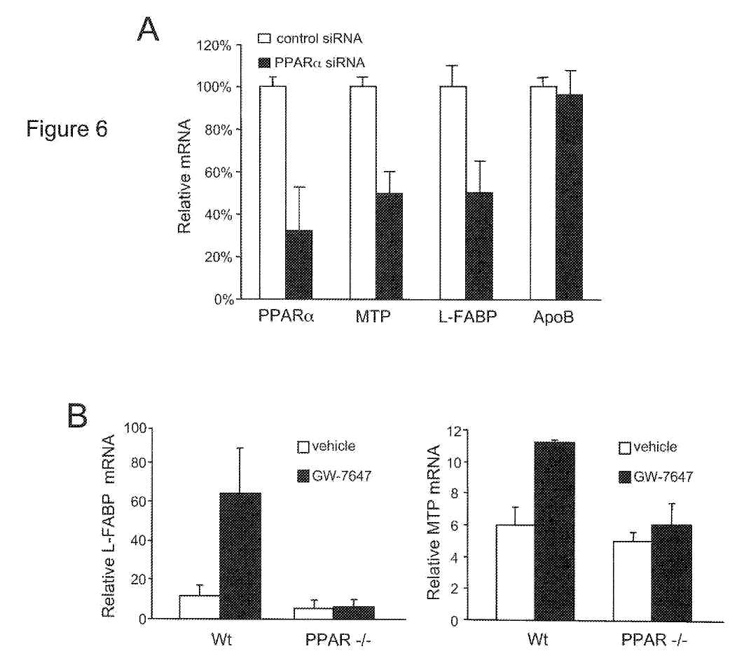

PPARα is necessary for high expression levels of L-FABP and MTP in hepatoma cells and in vivo. As such, the present invention predicts that the PPARα-RXRα activation complex is essential for the coordinated expression of L-FABP and MTP. To test this hypothesis, RNA interference was utilized to knockdown the expression levels of PPAR [symbol] in FAO cells. FAO cells transfected with PPARα-specific siRNAs demonstrated a 75% reduction PPARα mRNA compared to the cells transfected with control siRNA (FIG. 6A). This decrease in PPARα mRNA was associated with a reduced cellular content of both L-FABP and MTP mRNAs to nearly 50% of the control (FIG. 6A). The PPARα-specific RNAi did not alter the mRNA levels of PPARα-independent apoB, suggesting that, the decreases in L-FABP and MTP were PPARα-reduction specific (FIG. 6A). These findings indicate that PPAR [symbol] is necessary for the relatively high expression levels of L-FABP and MTP exhibited by FAO cells.

It was also examined whether a PPARα agonist would coordinately induce the hepatic expressions of L-FABP and MTP mRNAs in vivo and if so, whether the agonist-mediated increases were PPARα-dependent. Control C57/BL6 and PPARα−/− mice were treated with the PPARα agonist GW-7647 (28). While control mice displayed increased levels of both L-FABP and MTP mRNAs following treatment, with the PPARα agonist (FIG. 6B), mice lacking functional PPARα displayed no significant changes in either L-FABP and MTP expression levels (FIG. 6B). These in vivo data support the findings obtained using FAO/L35 hepatoma cells. Together the findings provided herein demonstrate the important role PPAR [symbol] plays in regulating the transcription of both L-FABP and MTP genes.

PGC-1β acts in concert with PPARα to coordinately induce the L-FABP and MTP Genes. The transcriptional coactivators PGC-1α and PGC-1β (29) exhibit distinct gene targets (16, 21). While PGC-1α activates genes involved in gluconeogenesis and mitochondrial biogenesis/fatty acid oxidation (21), PGC-1β activates genes involved in mitochondrial biogenesis/fatty acid oxidation and hepatic lipid transport (e.g. MTP (16)). The observations that L35 cells express relatively high levels of PGC-1 [symbol] and nearly undetectable levels of PGC-1β, whereas FAO cells express high levels of PGC-1β and nearly undetectable levels of PGC-1α (FIG. 7A) are consistent with the proposal that PGC-1β activates MTP gene expression (16). This proposal is further supported by the findings that show both L35 cells and mice treated with PPARα agonists caused a ˜3-fold induction of PGC-1β mRNA (FIG. 7B). To examine the role of PGC-1β in the induction of L-FABP and MTP, FAO cells were treated with PPARα-RXR [symbol] agonists and the effect of siRNA knockdown of PGC-1β was determined. PPARα-RXR [symbol] agonists treated FAO cells given the siRNA specific for PGC-1β demonstrated a 65% reduction in PGC-1β mRNA levels, which was associated, with correlative decreases in both L-FABP (−38%) and MTP (−48%) mRNA levels, whereas PGC-1α mRNA levels remained unchanged (FIG. 7C). PPARα-RXR [symbol] agonists treated FAO cells given a negative control siRNA exhibited no change in any of these mRNA levels (FIG. 7C). Thus, the siRNA demonstrated target-specificity and the associated reductions in L-FABP and MTP mRNA expressions were due to the reduction in PGC-1β content.

The role of PGC-1 [symbol] in L-FABP and MTP gene transcription was further examined by enhancing the expression of PGC-1 [symbol] in untreated and PPARα-RXR [symbol] agonist treated L35 cells via transfection with an adenovirus expressing PGC-1β (16, 30). The PGC-1β adenovirus did not affect L-FABP or MTP mRNA levels in untreated L35 cells (FIG. 8). In contrast, treating PPARα-RXR [symbol] agonist-stimulated L35 cells with the PGC-1β adenovirus significantly increased both L-FABP and MTP mRNAs (˜3 fold) (FIG. 8), relative to levels in both the agonist-treated uninfected and GFP-infected controls. Treating PPARα-RXR [symbol] agonist-stimulated L35 cells with an adenovirus that expressed GFP had no effect on the expression of L-FABP and MTP mRNAs (FIG. 8). These data provide additional support, for the proposal that both PPARα-RXRα and PGC-1β are necessary for enhancing the coordinated transcription of L-FABP and MTP.

Coordinate inactivation of L-FABP and MTP prevents hepatic steatosis in vivo. Retention of the functional DR1 site in both L-FABP and MTP promoters may allow coordinated, variation in expression and function (i.e. VLDL assembly/secretion). Chemical inhibition of MTP, a strategy developed to therapeutically ameliorate hyperlipidemia (31-34), is of limited use because of its association with the development of hepatic steatosis (22, 23). To examine if coordinate repression of L-FABP and MTP would block hepatic VLDL assembly/secretion without causing hepatic steatosis control and L-FABP null mice (L-FABP−/−) were treated with an MTP inhibitor (8aR) (23). After 7 days of 8aR treatment, plasma levels of triglyceride and cholesterol were markedly reduced to similar levels in both groups of mice (FIG. 9A). While treating control C57BL/6 mice with the MTP inhibitor resulted in a 4-fold increase in hepatic triglyceride levels, hepatic triglyceride accumulation was completely prevented by ablation of L-FABP (FIG. 9B). These data show that coordinate transcription regulation of L-FABP and MTP genes allows variation in VLDL assembly/secretion in the absence of hepatic steatosis.

### EXAMPLE 2

### Chemical Inhibition of L-FABP Prevents the Development of Hepatic Steatosis

This example illustrates that co-administration of a L-FABP inhibitor with a MTP inhibitor reduces plasma triglyceride concentrations.

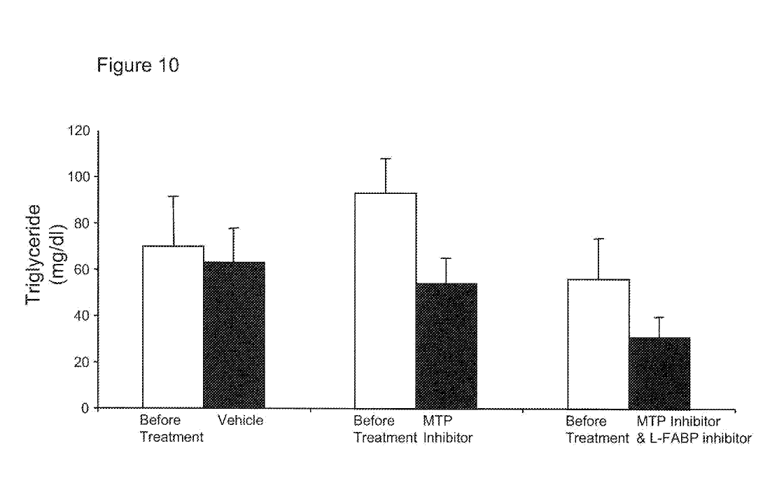

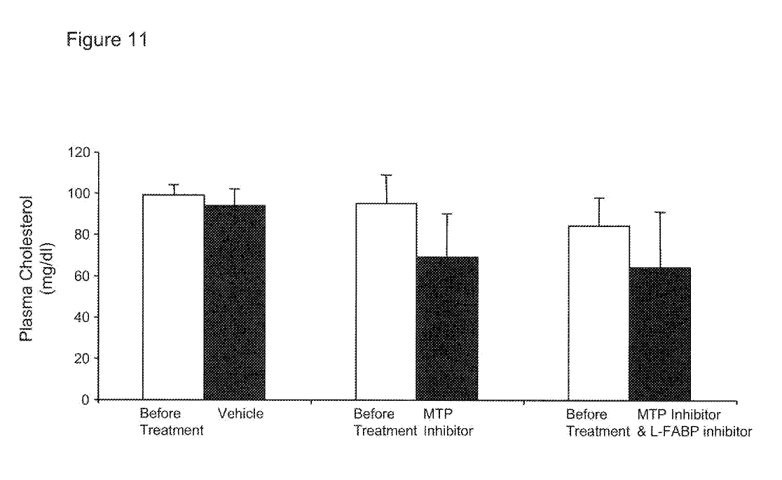

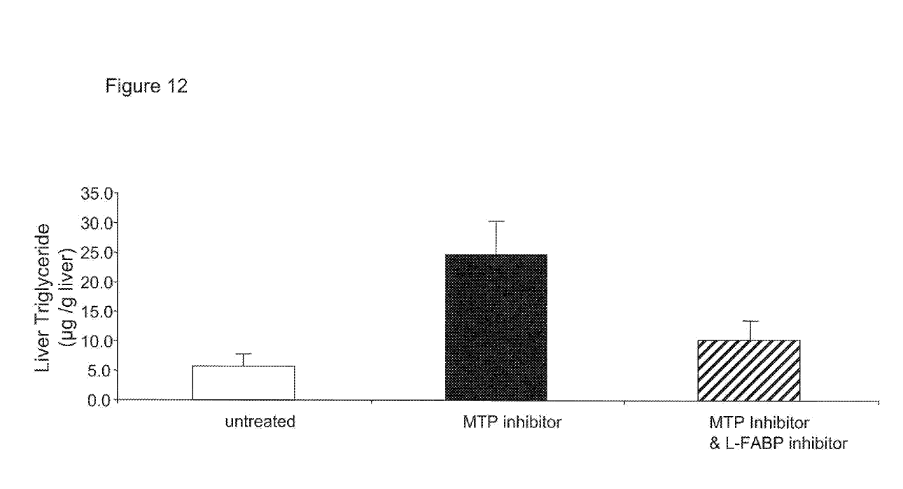

3-(decyldimethylsilyl)-N-[2-(4-methylphenyl)-1-phenylethyl]propanamide (Sandoz compound 58-035) (lot #fr. 09061988) has been shown to inhibit L-FABP. It was therefore examined if co-administration of Sandoz compound 58-035 with MTP inhibitor 8aR (Novartis, Summit, N.J.) would prevent the development of hepatic steatosis in wild-type mice. Prior to treatment, mice were bled and their serum, concentrations of triglycerides (FIG. 10) and cholesterol (FIG. 11) were measured. Mice were then gavaged with 0.150 ml of corn oil (vehicle only) or with corn oil containing the MTP inhibitor 8aR (50 mg/kg) or corn oil containing the MTP inhibitor 8aR (50 mg/kg) plus the L-FABP inhibitor Sandoz compound 58-035 (100 mg/kg). After 7 days of treatment, mice were bled and sacrificed. The MTP inhibitor reduced both plasma triglyceride levels (FIG. 10) and plasma cholesterol levels (FIG. 11) when administered with and without the L-FABP inhibitor. As expected, mice treated with the MTP inhibitor alone exhibited hepatic steatosis (liver triglyceride content was increased by 5-fold; FIG. 12). In marked contrast, livers from mice treated, with both the MTP inhibitor and the L-FABP inhibitor exhibited no increase in liver triglyceride content (FIG. 12). These data showing that co-administration of a L-FABP inhibitor with a MTP inhibitor prevents the development of hepatic steatosis extend the inventors' previous discovery that in mice lacking L-FABP expression due to targeted gene inactivation, MTP inhibitors do not cause fat accumulation. Moreover, these findings disclose that co-administration of agents capable of inhibiting L-FABP (i.e., Sandoz compound 58-035) enable MTP inhibitors to reduce plasma lipoproteins (FIGS. 10 and 11) without causing hepatic steatosis (FIG. 12).

Methods. C57BL/6 male mice (8-10 weeks old) (n=5 in each group) were having free access to drinking water and chow were gavaged daily with corn oil (0.15 ml) alone or corn oil containing the MTP inhibitor 8aR (50 mg/kg) (Novartis, Summit, N.J.) (Ksander, et al., 2001, Diaminoindanes as microsomal triglyceride transfer protein inhibitors. J Med Chem 44:4677-4687) or the MTP inhibitor 8aR (Novartis, Summit, N.J.) plus the L-FABP inhibitor Sandoz compound 58-035 (100 mg/kg) for 7 days. Mice were bled before beginning the experiment and after 7 days. Mice were housed in a room having a 12 hour light cycle (lights on from 6:00 AM to 6:00 PM). Mice were bled retro-orbitally at 10:00 AM and then sacrificed.

Liver triglyceride content and plasma content of cholesterol and triglycerides were determined as described (Liao, et al., 2003, Blocking microsomal triglyceride transfer protein interferes with apoB secretion without causing retention or stress in the ER. J Lipid Res 44:978-985). Values represent the mean±standard deviation of 5 mice in each group. *Denotes a significant difference between mice before and after treatment, p<0.05, Student's t test (FIG. 10).

FIG. 11 shows that co-administration of a L-FABP inhibitor with a MTP inhibitor reduces plasma cholesterol concentrations in mice. As above, C57BL/6 male mice (n= 5 in each group) were gavaged daily with corn oil alone or corn oil containing the MTP inhibitor 8aR (Novartis, Summit, N.J.) or the MTP inhibitor 8aR (Novartis, Summit, N.J.) plus the L-FABP inhibitor Sandoz compound 58-035. Mice were bled before beginning the experiment and after 7 days. Plasma content of cholesterol was determined as described. Values represent the mean±Standard deviation of 5 mice in each group.

FIG. 12 shows that co-administration of a L-FABP inhibitor with a MTP inhibitor prevents the development of hepatic steatosis in mice. As above, C57BL/6 male mice (n=5 in each group) were gavaged daily with corn oil alone or corn oil containing the MTP inhibitor 8aR (Novartis, Summit, N.J.) or the MTP inhibitor 8aR (Novartis, Summit, N.J.) plus the L-FABP inhibitor Sandoz compound 58-035. Mice were sacrificed after 7 days. Liver triglyceride content was determined. Values represent the mean±standard deviation of 5 mice in each group.

Although the invention has been described with reference to the above example, it will be understood that modifications and variations are encompassed within the spirit and scope of the invention. Accordingly, the invention is limited only by the following claims.

### REFERENCES

1. Mann, C. J., Anderson, T. A., Read, J., Chester, S. A., Harrison, G. B. Kochl, S., Ritchie, P. J., Bradbury, P., Hussain, F. S., Amey, J., et al. 1999. The Structure of Vitellogenin Provides a Molecular Model for the Assembly and Secretion of Atherogenic Lipoproteins. J Mol Biol 285:391-408.
2. Shelness, G. S., and Ledford, A. S. 2005, Evolution and mechanism, of apolipoprotein B-containing lipoprotein assembly. Curr Opin Lipidol 16:325-332.
3. Hui, T. Y., Olivier, L. M., Kang, S., and Davis, R. A. 2002. Microsomal triglyceride transfer protein is essential for hepatic secretion of apoB-100 and apoB-48 but not triglyceride, J Lipid Res 43:785-793.
4. Kang, S., Spann, N J., Hui, T. Y., and Davis, R. A. 2003. ARP-1/COUP-TF II determines hepatoma phenotype by acting as both a transcriptional repressor of microsomal triglyceride transfer protein and an inducer of CYP7A1. J Biol Chem 278:30478-30486.
5. Scarino, M. L., and Howell, K. E. 1987. The Fao cell, A tissue culture model for lipoprotein synthesis and secretion. II. Modulation by lipid depletion and supplementation. Exp Cell Res 170:15-30.
6. Scarino, M. L., and Howell, K. E. 1987. The Fao cell. A tissue culture model for lipoprotein synthesis and secretion. II. Characterization of the system. Exp Cell Res 170:1-14.
7. Phelps, C, Gburcik, V., Suslova, E., Dudek, P., Forafonov, F., Bot, N., Maclean, M., Fagan, R. J., and Picard, D. 2006. Fungi and animals may share a common ancestor to nuclear receptors, Proc Natl Acad Sci USA 103:7077-7781.
8. Wall M. E., Dunlop, M. J., and Hlavacek, W. S. 2005. Multiple Functions of a Feed-Forward-Loop Gene Circuit. Journal of Molecular Biology 349:501-514.
9. Poirier, H., Niot, I., Monnot, M. C., Braissant, O., Meunier-Durmort, C., Costet, P., Pineau, T., Wahli, W., Willson, T. M., and Besnard, P. 2001, Differential involvement of peroxisome-proliferator-activated receptors alpha and delta in fibrate and fatty-acid-mediated inductions of the gene encoding liver fatty-acid-binding protein in the liver and the small intestine. Biochem J 355:481-488.
10. Ameen, C, Edvardsson, U., Ljungberg, A., Asp, L., Akerblad, P., Tuneld, A., Olofsson, S. O., Linden, D., and Oscarsson, J. 2005. Activation of peroxisome proliferator-activated receptor alpha increases the expression and activity of microsomal triglyceride, transfer protein in the liver. J Biol Chem 280:1224-1229.
11. Poirier, H., Braissant, O., Niot, I., Wahli, W., and Besnard, P. 1997. 9-cis-retinoic acid enhances fatty acid-induced expression Of the liver fatty acid-binding protein gene, FEBS Lett 412:480-484.
12. Miyata, K. S., Zhang, B., Marcus, S. L., Capone, J. P., and Rachubinski, R. A. 1993. Chicken ovalbumin upstream promoter transcription factor (COUP-TF) binds to a peroxisome proliferator-responsive element and antagonizes peroxisome proliferator-mediated signaling. J Biol Chem 268:19169-19172.
13. Carter, M. E., Gulick, T., Moore, D. D., and Kelly, D. P. 1994. A pleiotropic element in the medium-chain acyl coenzyme A dehydrogenase gene promoter mediates transcriptional regulation by multiple nuclear receptor transcription factors and defines novel receptor-DNA binding motifs. Mol Cell Biol 14:4360-4372.
1.4. Cabrera, A., Jove, M., Planavila, A., Merlos, M., Laguna, J. C., and Vazquez-Carrera, M. 2003, Down-regulation of acyl-CoA oxidase gene expression in heart of troglitazone-treated mice through a mechanism involving chicken ovalbumin upstream promoter transcription factor II. Mol Pharmacol 64:764-772.
15. Palmer, C. N., Hsu, M. H., Muerhoff, A. S., Griffin, K. J., and Johnson, E. F. 1994. Interaction of the peroxisome proliferator-activated receptor alpha with the retinoid X receptor alpha unmasks a cryptic peroxisome proliferator response element that overlaps an ARP-1-binding site in the CYP4A6 promoter, J Biol Chem 269:18083-18089.
16. Lin, J., Yang, R., Tarr, P. T., Wu, P. H, Handschin, C., Li, S., Yang, W., Pei, L., Uldry, M., Tontonoz, P., et al. 2005. Hyperlipidemic effects of dietary saturated fats mediated through PGC-1beta coactivation of SREBP. Cell 120:261-273.
17. Wolfrum, C. and Stoffel, M. 2006. Coactivation of Foxa2 through Pgc-1beta promotes liver fatty acid oxidation and triglyceride/VLDL secretion. Cell Metab 3:99-110.
18. Wolfrum, C, Asilmaz, E., Luca, E., Friedman, J. M., and Stoffel, M. 2004. Foxa2 regulates lipid metabolism and ketogenesis in the liver during fasting and in diabetes. Nature 432:1027-1032.
19. Bartels, E. D., Lauritsen, M., and Nielsen, L. B. 2002. Hepatic expression of microsomal triglyceride transfer protein and in vivo secretion of triglyceride-rich lipoproteins are increased in obese diabetic mice. Diabetes 51:1233-1239.
20. Lin, J., Wu, H., Tarr, P. T., Zhang, C. Y., Wu, Z., Boss, O., Michael, L. F., Puigserver, P., Isotani, E., Olson, E. N., et al. 2002. Transcriptional co-activator PGC-1 alpha drives the formation of slow-twitch muscle fibres. Nature 418:797-801.
21. Lin, J., Tarr, P. T., Yang, R., Rhee, J., Puigserver, P., Newgard, C. B., and Spiegelman, B. M. 2003, PGC-1beta in the regulation of hepatic glucose and energy metabolism. J Biol Chem 278:30843-30848.
22. Bjorkegren, J., Beigneux, A., Bergo, M. O., Maher, J. J., and Young, S. G., 2002. Blocking the secretion of hepatic very low density lipoproteins renders the liver more susceptible to toxin-induced injury. J Biol Chem 277:5476-5483.
23. Liao, W., Hui, T. Y., Young, S. G., and Davis, R. A. 2003. Blocking microsomal triglyceride transfer protein interferes with apoB secretion without causing retention or stress in the ER. J Lipid Res 44:978-985.
24. Leighton, J. K., Joyner, J., Zamarripa, J., Deines, M., and Davis, R. A. 1990, Fasting decreases apolipoprotein B mRNA editing and the secretion of small molecular weight apoB by rat hepatocytes: Evidence that the total amount of apoB secreted is regulated post-transcriptionally, J Lipid Res 31:1663-1668.
25. Newberry, E. P., Xie, Y. Kennedy, S., Han, X., Buhman, K. K., Luo, J., Gross, R. W., and Davidson, N. O. 2003. Decreased hepatic triglyceride accumulation and altered fatty acid uptake in mice with deletion of the liver fatty acid-binding protein gene. J Biol Chem 278:51664-51672.
26. Borchardt, R. A., and Davis, R. A. 1987. Intrahepatic assembly of very low density lipoproteins. Rate of transport out of the endoplasmic reticulum determines rate of secretion. J. Biol. Chem. 262:16394-16402.
27. Pauley, C. J., Ledwith, B. J., and Kaplanski, C. 2002. Peroxisome proliferators activate growth regulatory pathways largely via peroxisome proliferator-activated receptor alpha-independent mechanisms, Cell Signal 14:351-358.
28. Fu, J., Oveisi, F., Gaetani, S., Lin, E., and Piomelii, D. 2005. Oleoylethanolamide, an endogenous PPAR-alpha agonist, lowers body weight and hyperlipidemia in obese rats. Neuropharmacology 48:1147-1153.
29. Kressler, D., Schreiber, S. N., Knutti, D., and Kralli, A. 2002. The PGC-1-related Protein PERC Is a Selective Coactivator of Estrogen Receptor alpha. J. Biol. Chem. 277:13918-13925.
30. Lin, J., Puigseryer, P., Donovan, J., Tarr, P., and Spiegelman, B. M. 2002. Peroxisome proliferator-activated receptor gamma coactivator 1beta (PGC-1beta), a novel PGC-1-related transcription coactivator associated with host cell factor. J Biol Chem 277:1645-1648.
31. Wetterau, J. R., Gregg, R. E., Harrity, T. W., Arbeeny, C, Cap, M Connolly, F., Chu, C. H., George, R. J., Gordon, D. A., Jamil, H., et al. 1998. An MTP inhibitor that normalizes atherogenic lipoprotein levels in WHHL rabbits. Science 282:751-754.
32. Chandler, C. E., Wilder, D. E., Pettini, J. L., Savoy, Y. E., Petras, S. F., Chang, G., Vincent, J., and Harwood, H. J., Jr. 2003. CP-346086: an MTP inhibitor that lowers plasma cholesterol and triglycerides in experimental animals and in humans. J Lipid Res 44:1887-1901.
33. Ksander, G. M., deJesus, R., Yuan, A., Fink, G, Moskal, M., Carlson, E., Kukkola, P., Bilci, N., Wallace, E., Neubert, A., et al. 2001. Diaminoindanes as microsomal triglyceride transfer protein inhibitors. J Med Chem 44:4677-4687.
34. Shiomi, M., and Ito, T. 2001. MTP inhibitor decreases plasma cholesterol levels in LDL receptor-deficient WHHL rabbits by lowering the VLDL secretion. Eur J Pharmacol 431:127-131.
35. Frolov, A., Miller, K., Billheimer, J. T., Cho, T. H., and Schroeder, F. 1997, Lipid specificity and location of the sterol carrier protein-2 fatty acid-binding site: a fluorescence displacement and energy transfer study. Lipids 32:1201-1209.
36. Frolov, A., Cho, T. H., Murphy, E. J., and Schroeder, F. 1997. Isoforms of rat liver fatty acid binding protein differ in structure and affinity for fatty acids and fatty acyl CoAs. Biochemistry 36:6545-6555.
37. Woodford, J. K., Behnke, W. D., and Schroeder, F. 1995. Liver fatty acid binding protein enhances sterol transfer by membrane interaction. Mol Cell Biochem 152:51-62.
38. Schroeder, F Myers-Payne, S. C., Billheimer, J. T., and Wood, W. G. 1995. Probing the ligand binding sites of fatty acid and sterol carrier proteins: effects of ethanol. Biochemistry 34:11919-11927.
39. Schroeder, F., Hubbell, T., Colies, S. M., and Wood, W. G. 1995. Expression of liver fatty acid binding protein in L-cells: plasma membrane response to ethanol. Arch Biochem Biophys 316:343-352.
40. Hubbell, T., Behnke, W. D., Woodford, J. K., and Schroeder, F. 1994, Recombinant liver fatty acid binding protein interacts with fatty acyl-coenzyme A. Biochemistry 33:3327-3334.
41. Woodford, J. K., Jefferson, J. R., Wood, W. G., Hubbell, T., and Schroeder, F. 1993. Expression of liver fatty acid binding protein alters plasma membrane lipid composition and structure in transfected L-cell fibroblasts. Biochim Biophys Acta 1145:257-265.
42. Schroeder, F., Jefferson, J. R., Powell, D., Incerpi, S., Woodford, J. K., Colles, S. M., Myers-Payne, S., Emge, T., Hubbell, T., Moncecchi, D., et al. 1993. Expression of rat L-FABP in mouse fibroblasts: role in fat absorption. Mol Cell Biochem 123:73-83.
43. Nemecz, G., Hubbell, T., Jefferson, J. R., Lowe, J. B., and Schroeder, F. 1991. Interaction of fatty acids with recombinant rat intestinal and liver fatty acid-binding proteins. Arch Biochem Biophys 286:300-309.
44. Jamil, H., Chu, C. H., Dickson, J. K., Jr., Chen, Y., Yan, M., Biller, S. A., Gregg, R. E., Wetterau, J. R., and Gordon, D. A. 1998. Evidence that microsomal triglyceride transfer protein is limiting in the production of apolipoprotein B-containing lipoproteins in hepatic cells. J Lipid Res 39:1448-1454.
45. Raabe, M., Flynn, L. M., Zlot, C. H., Wong, J. S., Veniant, M. M., Hamilton, R. L., and Young, S. G. 1998. Knockout of the abetalipoproteinemia gene in mice: reduced lipoprotein secretion in heterozygotes and embryonic lethality in homozygotes. Proc Natl Acad Sci USA 95:8686-8691.
46. Fleming, J. F., Spitsen, G. M., Hui, T. Y., Olivier, L., Du, E. Z., Raabe, M., and Davis, R. A. 1999. Chinese hamster ovary cells require the coexpression of microsomal triglyceride transfer protein and cholesterol 7alpha-hydroxylase for the assembly and secretion of apolipoprotein B-containing lipoproteins, J Biol Chem 274:9509-9514.
47. Leung, G. K., Veniant, M. M., Kim, S. K., Zlot, C. H., Raabe, M., Bjorkegren, J., Neese, R. A., Hellerstein, M. K., and Young, S. G. 2000. A deficiency of microsomal triglyceride transfer protein reduces apolipoprotein B secretion, J Biol Chem 275:7515-7520.
48. Jamil, H., Gordon, D. A., Eustice, D. C., Brooks, C. M., Dickson, J. J., Chen, Y., Ricci, B., Chu, C. H., Harrity, T. W., Ciosek, C. J., et al. 1996. An inhibitor of the microsomal triglyceride transfer protein inhibits apoB secretion from HepG2 cells. Proc Natl Acad Sci USA 93:11991-11995.
49. Robl, J. A., Sulsky, R., Sun, C. Q., Simpkins, L. M., Wang, T., Dickson, J. K., Jr., Chen, Y., Magnin, D. R., Taunk, P., Siusarchyk, W. A., et al. 2001. A novel series of highly potent benzimidazole-based microsomal, triglyceride transfer protein inhibitors. J Med Chem 44:851-856.

What is claimed is:

1. A pharmaceutical compound made according to the process comprising:
providing a first agent that has the ability to inhibit Microsomal Triglyceride Transfer Protein (MTP);
providing a second agent that has the ability to inhibit Liver Fatty Acid-Binding Protein (L-FABP);
covalently bonding said first agent and said second agent thereby creating said pharmaceutical compound.

2. The pharmaceutical compound of claim 1, wherein said covalently bonding permits said pharmaceutical compound to (a) be absorbed by the intestine intact, and (c) achieve physiological activation in the liver.

3. The pharmaceutical compound of claim L wherein said covalently bonding comprises a bond is selected from the group consisting of carboxylic esters, phospho-esters, amities (—R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof.

4. The pharmaceutical compound of claim 1, wherein said pharmaceutical compound is capable of inhibiting MTP and L-FABP in vivo.

5. The pharmaceutical compound of claim 4, wherein said pharmaceutical compound is effective in the treatment of hyperlipidemia.

6. A method for identifying a candidate composition for the treatment of hyperlipidemia comprising:
identifying a first agent capable of inhibiting Microsomal Triglyceride Transfer Protein (MTP);
identifying a second agent capable of inhibiting Liver Fatty Acid-Binding Protein (L-FABP);
identifying the combination of said first agent and said second agent as a candidate composition for use in the treatment of hyperlipidemia.

7. Tire method of claim 6, wherein said combination comprises a single molecule wherein said first agent and said second agent are linked by covalent bonding.

8. The method of claim 7, wherein said covalently bonding permits said pharmaceutical compound to (a) be absorbed by the intestine intact, and (c) achieve physiological activation in the liver.

9. The method of claim 7, wherein said covalent bonding comprises a bond selected from the group consisting of carboxylic esters, phospho-esters, amides (—R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof.

10. A method for treating hyperlipidemia comprising:
administering, to a patient in need thereof, an effective amount of a composition that inhibits Microsomal Triglyceride Transfer Protein (MTP) and Liver Fatty Acid-Binding Protein (L-FABP).

11. The method of claim 10, wherein said composition comprises a pharmaceutical compound comprising a first agent that inhibits MTP, and a second agent that inhibits L-FABP.

12. The method of claim 11, wherein said first agent and said second agent are linked by covalent bonding.

13. The method of claim 12, wherein said covalent bonding permits said composition to (a) be absorbed by the intestine intact, and (c) achieve physiological activation in the liver.

14. The method of claim 12, wherein said covalent bonding comprises a bond selected from the group consisting of carboxylic esters, phospho-esters, amides (—R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof.

15. A pharmaceutical compound made according to the process comprising:
identifying a first agent capable of inhibiting Microsomal Triglyceride Transfer Protein (MTP);
identifying a second agent capable of inhibiting Liver Fatty Acid-Binding Protein (L-FABP);
forming said pharmaceutical compound by covalently bonding said first agent to said second agent.

16. The pharmaceutical compound made according to the process of claim 1, wherein said pharmaceutical compound is capable of (a) being absorbed by the intestine intact, and (b) achieving physiological activation in the liver

17. The pharmaceutical compound made according to the process of claim 1, wherein said covalently bonding comprises a bond selected from the group consisting of carboxylic esters, phospho-esters, amides (—R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof.

18. The pharmaceutical compound made according to the process of claim 1, wherein said pharmaceutical compound is capable of inhibiting MTP and L-FABP in vivo.

19. The pharmaceutical compound made according to the process of claim 1, wherein said pharmaceutical compound is capable of treating hyperlipidemia.

20. A pharmaceutical compound comprising:
a first agent that inhibits Microsomal Triglyceride Transfer Protein (MTP);
a second agent that inhibits Liver Fatty Acid-Binding Protein (L-FABP); and
a covalent bond linking said first agent and said second agent.

21. The pharmaceutical compound of claim 20, wherein said covalent bond permits said pharmaceutical compound to (a) be absorbed by the intestine intact, and (b) achieve physiological activation in the liver.

22. The pharmaceutical compound of claim 20, wherein said covalent bond comprises a bond selected from the group consisting of carboxylic esters, phospho-esters, amides (R—N(H)—C(O)—R′, disulfides (R—S—S—R′), sulfo-esters (R—O—S(O)—R′), sulfoxide-esters (R—O—S(O)—O—R′), peroxides (R—O—O—R′), and combinations thereof.

23. The pharmaceutical compound of claim 20, wherein said compound is effective in the treatment of hyperlipidemia.

24. A pharmaceutical compound comprising:
a first agent that inhibits Microsomal Triglyceride Transfer Protein (MTP);
and a second agent that inhibits Liver Fatty Acid-Binding Protein (L-FABP);
wherein said first pharmaceutical active and said second pharmaceutical active are joined by covalent bonding.

25. The pharmaceutical compound of claim 24, wherein said first agent and said second agent are small molecules.
